(19) United States
(12) Patent Application Publication
(10) Pub. No.: US 2014/0243608 A1
(43) Pub. Date: Aug. 28, 2014

(54) SYSTEMS AND METHODS FOR CLINICAL EVALUATION OF PSYCHIATRIC DISORDERS

(71) Applicant: Robert D. Hunt, Nashville, TN (US)

(72) Inventor: Robert D. Hunt, Nashville, TN (US)

(21) Appl. No.: 14/130,772

(22) PCT Filed: Jul. 5, 2012
(86) PCT No.: PCT/US2012/045562

§ 371 Date: Apr. 3, 2014

Related U.S. Application Data
(60) Provisional application No. 61/504,677, filed on Jul. 5, 2011.
(60) Provisional application No. 61/515,765, filed on Aug. 5, 2011.

(51) Int. Cl. A61B 5/00; A61B 5/16; A61B 5/0476

(52) U.S. Cl. CPC A61B 5/4848; A61B 5/0042; A61B 5/0476; A61B 5/0022; A61B 5/7282; A61B 5/168; A61B 5/165; A61B 5/4088; A61B 5/6898
USPC 600/300

(57) ABSTRACT

A method of optimizing the effectiveness of a medication for the treatment of a neurobehavioral or psychiatric disorder in a patient is presented. Medication is administered to a patient. Patient information is obtained and stored in a computer database. Real-time current information is received, for example from a mobile computer device, by the patient relating to (i) medication being taken by the patient; (ii) at least one benefit or lack of benefit of the medication to the patient; (iii) characteristics of one or more current tasks being performed by the patient; and (iv) a current condition of the patient. The effectiveness of the medication is evaluated based on stored patient information and received real-time current information. Accordingly, the dosage or frequency of the medication administered to the patient may be modified, eliminated or maintained.

### CROSS-REFERENCE TO RELATED APPLICATIONS

This application is a 371 National Stage Application on PCT/US2012/045562, filed Jul. 5, 2012, which claims priority to U.S. 61/504,677, filed Jul. 5, 2011 and 61/515,765, filed Aug. 5, 2011, the entire contents of which are incorporated herein by reference.

### BACKGROUND

Many psychiatric disorders that occur in children and adults are treatable with various medications. The U.S. Food and Drug Administration (FDA) has approved medications that are effective for the treatment of major psychiatric disorders, such as an Attention-Deficit Hyperactivity Disorder (e.g., ADHD and/or ADD), mood disorders (e.g., depression or bipolar), anxiety disorders (e.g., Obsessive Compulsive Disorder (OCD), Post Traumatic Stress Disorders (PTSD) and social anxiety), and thought disorders (e.g., Aspergers or psychosis) or substance abuse. For example, for the treatment of ADD/ADHD, the FDA has approved at least two major classes of medications with distinct neurochemical mechanisms of action (amphetamines and methylphenidate), with several variations in each class based on differences in mechanisms of release that impact duration of action. Recently FDA has extended approval to noradrenergic agents including guanfacine (Intuniv) and atomoxetine (Strattera). Similarly, with antidepressants, the FDA has approved a large spectrum of medications with varied neurochemical effects for use in the treatment of depression.

FDA approval typically relates to a single medication for the treatment of a single psychiatric diagnosis. Likewise, current psychopharmacological research nearly always study patients having only one diagnosis, who are treated with only one medication, compared to placebo. Such studies typically exclude patients with multiple diagnoses. Patients suffering from psychiatric disorders, however, often have multiple psychiatric problems that cross several diagnostic categories. Moreover, diagnostics relationships between different psychiatric disorders are complex and interactive. Thus, physicians often do not have an adequate guide for patient specificity and optimal clinical treatment. Very little guidance exists to help clinicians treat patients that present multiple simultaneous diagnoses and require more complex treatments. In addition, very little guidance is available to help clinicians account for variations in the personalities of patients and/or in their metabolic and behavioral responses to psychiatric drugs.

Further, almost no systemic attention has been paid to the differences in the type or quality of attention required for specific tasks, and the relevance of this to specific medications. Some tasks require frequent shifting from one component to another—all of which may be familiar to the patient. Most adults spend much of their time doing tasks they are already well trained for. They predominantly require wide, flexible attention. However, other circumstances require significant new learning and significant study and concentration. Applicant's research suggests that specific psychostimulants are preferable for specific types of tasks and learning. For example, Vyvanse (d-amphetamine with a prolonged release mechanism) is excellent for shifting attention, while Adderall (combined d- & l-amphetamine) works well for depth of attention needed for new learning. Concerta (a long-acting methylphenidate) is more inhibiting and better for children who need to sit and be quiet in a classroom environment. Such medications vary greatly in their duration of action. Some medications may be administered once daily, while others require repeated administration. Differences in patient tasks, energy level, and metabolism, for example, are relevant to a “Best Fit” optimization.

Insurance companies are acutely aware of the waste and inefficiencies inherent in the health care system that arise from many patients receiving medications that are not appropriately tailored to the patient's needs. Unfortunately, an accurate mechanism for establishing clinical specificity, i.e., a “Best Fit” verifiable model for predicting optimal matching of medication to a patient, has been missing from standard treatment protocols. The absence of an effective model based on objective and subjective data leads to the suboptimal or incorrect and inadequate treatment of many patients, adding considerable cost and waste to the health care system. Patients are often randomly shifted from one psychostimulant to another with little rationale for making these changes. This attendant cost inefficiency is borne by patients, insurance companies and by state and national health care. Further, patients frequently cease needed treatment because they experience their medication as being ineffective or uncomfortable.

### SUMMARY

Conventional techniques often survey a patient (e.g., during a doctor's appointment) at a time of day removed from treatment and/or tasks normally performed by a patient. In fact, the physician's decision is generally based on a quick response to very general questions, such as “Do you think this medication is working?” or “Are you experiencing any Side Effects?”. There is little attention to the actual tasks a patient performs in their life, the varied attentional demand of these tasks, and the medication and dose specificity required for optimization. The common presumption is that all stimulants work about the same, but they differ predominately in their duration of action. Applicant's experience with many hundreds of patients interviewed in detail suggests that different stimulants vary significantly in their facilitation of flexible attention, sustained attention, and enhancement of new learning. They also differ in their impact on energy—alertness and arousal that impacts cognitive processing including memory storage and retrieval and capacity for problem-solving and innovation Such techniques do not accurately capture benefits and side effects of treatment because they rely on the patient's memory and vocabulary/ability to describe how they were affected by the medication. Contrary to conventional understanding and techniques, a need exists for ways to accurately evaluate benefits, side effects and other effects of a patient's medication, as they occur in real-time. As described herein, Applicant has designed a system, such as one using a computer system linked to a mobile device, such as a smart phone (e.g., Apple's iPhone®, Android® phones, etc.), tablets (e.g., Apple's iPad®, HP's TouchPad®, Samsung and Google tablets, etc.), and laptops, etc., for obtaining and evaluating accurate, real-time clinical data documenting the benefits, limitations, and side effects of a prescribed medication. The present invention takes into account that companies are coming out with alternative tablets, such as HP's TouchPad®. As such, the present invention contemplates, for example, a Mac or PC format that runs on a computer via typing, a touch pad or touch screen via stylus or manual touch.

Applicant's real-time clinical evaluation system provides a mechanism to create a “Best Fit” that allows physicians to optimize the efficacy of medication for individual patients. The phrase “Best Fit” refers to a treatment protocol that works in the best way, at the best time, to optimally help a patient perform the specific tasks most relevant to that patient. With respect to short-acting medications, such as psychostimulant medications, the ability to evaluate the efficacy of the prescribed medication in real-time adds a critical time variable for determining the best medication, dosage, and delivery mechanism (“Best Fit”) for a given patient. Some medications, such as Vyvanse and Concernta last about 12 hrs. Others, such as Adderall XR and Focalin XR last about 8 hrs. Regular Adderall, Ritalin and Focalin last 2-4 hrs. In addition there exists considerable individual patient variability in the parameters of efficacy and duration. The difference may be predominately pharmacokinetic (differences in rate of absorption, metabolism and excretion) and/or pharmacodynamic (differences in receptor sensitivity to medication effects on relevant neurotransmitters, predominately dopamine and norepinephrine). In fact, some aspects of duration and clinical response change over time with exposure to medication. The dose may need to be increased after a couple months of treatment. Task and personality factors are also relevant. Applicant's evaluation system extends beyond a single diagnosis to incorporate multiple diagnosis, personality characteristics, neurobiological variables, task variables and medication variables. Applicant's “Best Fit” model takes into account multiple factors at once, such as (1) diagnoses; (2) medication taken by the patient, such as information relating to type, amount, dosages, administration times of day; (3) tasks being performed by the patient, such as objective and subjective information relating to attentional demand of a task; (4) environment, such as distractions, motivational reinforcements, and subjective impact; (5) personality characteristics; and (6) metabolic and pharmacodynamic factors.

Personality characteristics, tasks, environments, for example, have not been previously applied in a systematic model for defining a “Best Fit” treatment. Yet, such aspects impact a patient's response to and preference for, and compliance with medication treatment. Although differences in personality may be less critical than differences in diagnoses and the presence of simultaneous concurrent or multiple diagnoses, they are clearly relevant. Applicant's system can account for personality characteristics/factors/traits in a manner that is simple, empirically defined and easily measured.

One major problem with medication treatment of psychiatric disorders is compliance by the patient with the prescription regimen in a manner that sustains treatment. ADD is a life-long disorder with continued impact on productivity and efficiency. Yet 40% of patients stop their medication within 4 months, and 60% discontinue after 6 months. This high incidence of discontinuation makes the concept of preference relevant to the treatment of ADD. Applicant's system can account for, or improve, compliance and/or issues related to compliance.

Neurobiological variables that a “Best Fit” model may consider are continuously expanding and can include molecular genetics, brain imaging (PET, MRI, and SPECT scans), electrical activity in the brain using Q-EEG, etc. Previously, relatively little systematic research has been done on how to use such information in the selection of medication and/or other interventions with definitive measures of clinical response—relevant to enhancing “Best Fit” for specific treatments.

Stimulant medications have distinct differences that affect their impact on the performance of different types of tasks (as measured by task complexity, variety, and degree of shifting (multitasking)) which can be measured at specific times during the day. The system described herein easily and efficiently allows a patient to enter a description of what task they are doing at a specific time—and how much attention is required to perform these tasks at that moment. Applicant's system can link the cognitive demands of the patient's tasks to the clinical characteristics of specific medications to enhance the “Best Fit” of treatment in relation to task. In addition, regarding psychostimulant medications, Applicant's system can account for clinically significant differences in chemical composition, mechanism of action, duration of effect, and impact on thinking and task performance.

Factors considered in a “Best Fit” model may also include diagnoses (which in many patients are not singular) and their impact on specific aspects of productivity and efficiency in relation to the specific type of tasks being performed. Applicant's model recognizes both the objective and descriptive aspects of the task (e.g., what the patient is doing, how complex is the task, how flexible is the task, etc.) and subjective aspects of the task (the patient's interest level, familiarity with, and attitude towards the task). Applicant's system also considers relevant environmental factors, including ambient factors (degree of distractions and interruptions), and motivational factors (degree of rewards, reinforcements, and emotional incentives (appreciation, recognition and encouragement) and the patient's own subjective investment (interest and motivation) in the work they do.

Clinically, the “Best Fit” can match the duration of medications to the time and duration of tasks being performed. A “Best Fit” protocol helps the prescribing physician select the right medication for an individual patient, at the right dose, at the right time, for the actual task(s) being performed by that patient. It collectively takes into account the effects of Task×Drug×Dose×Time, as quantifiable by, for example, one or more equipped computers and/or mobile devices. A “Best Fit” scenario may differ for a child in elementary school, a college student studying for a final exam, or a business executive preparing for an annual shareholders meeting.

The information obtained by the system is designed through built-in analytical methods to generate an index of the effectiveness of the medication, the duration of this effect, the need for additional doses to maximize or sustain effect, or the probability that a change of medication would be more likely to be more effective or to sustain efficacy longer. This integration and recommendation incorporates measures of the type of tasks the patient performs, their relative pattern of attentional demand (wide, or narrow, flexible or sustained, detailed or conceptual) as relavant factors in selection of medication, dose, and timing. Although this process currently is based on clinical experience, it will become self-informing and continuously improving. The system is anticipated to be used by hundreds and eventually thousands of patients, constituting a vast database for continuous refinement of implications and recommendations.

It should be appreciated that all combinations of the foregoing concepts and the additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated the terminology explicitly employed herein that also may appear in any disclosure incorporated by reference should be accorded a meaning most consistent with the particular concepts disclosed herein.

### BRIEF DESCRIPTION OF THE DRAWINGS

The inventive subject matter disclosed herein may be best understood by reference to the following description, taken in connection with the accompanying drawings as set forth below. The drawings are not necessarily to scale, emphasizing instead generally being placed upon illustrating the principles of the various inventive embodiments.

FIG. 1 illustrates a functional block diagram of a real-time clinical evaluation system, according to one embodiment of the present invention.

FIGS. 2A and 2B illustrate a mobile device and a corresponding functional block diagram, according to one embodiment of the present invention to facilitate the real-time capture of patient-specific data needed to evaluate the benefits, other effects, or side effects of a medication for the treatment of a patient.

FIGS. 3A-3C illustrate a central computer and a corresponding functional block diagram to facilitate real-time clinical evaluation of the benefits, other effects, or side effects of a medication for the treatment of a disorder in a patient, according to one embodiment of the present invention

FIGS. 3D-3I illustrate information that can be stored in a real-time clinical evaluation database according to one embodiment of the present invention.

FIG. 4A illustrates a flow chart of a method for real-time clinical evaluation of the effects of a medication for the treatment of a disorder in a patient, according to one embodiment of the present invention.

FIG. 4B illustrates a block diagram of acquired patient information, according to one embodiment of the present invention.

FIG. 5A illustrates a flow chart of a method for real-time clinical evaluation of the benefits, other effects, or side effects of a medication for the treatment of a disorder in a patient, according to one embodiment of the present invention.

FIG. 5B illustrates a diagram for evaluating patient information, according to one embodiment of the present invention.

FIGS. 6-39 illustrate screen shots of a user interface for a real-time clinical evaluation system, according to one embodiment of the present invention.

FIG. 40 is a diagram illustrating three axes of personality factors, i.e., dependence, harm avoidance, and novelty seeking, according to one embodiment of the present invention.

FIGS. 41-43 are diagrams illustrating the effectiveness of one example type of medication, according to one embodiment of the present invention.

### DETAILED DESCRIPTION

Various embodiments of the present invention relate to a system, and associated methods and apparatus, for evaluating benefits, other effects and/or side effects of a medication for the treatment of a disorder in a patient, as evaluated in real-time.

In particular, the systems, methods and apparatus according to various embodiments disclosed herein provide communication infrastructure, software applications and computing devices, and various instruments and methods for evaluating benefits, other effects and/or side effects of a medication for the treatment of a disorder in a patient in real-time.

In various exemplary implementations, one or more aspects of the evaluation systems, methods and apparatuses employ automated applications and instruments for electronically processing and/or analyzing information relevant to clinical evaluation.

### DEFINITIONS

The term “real-time” in relation to evaluating benefits and/or effects of medication or an evaluation system refers to an evaluation or system that updates information at the same time and rate such information is received. Information provided or received in “real-time” means that information is provided or received contemporaneously or nearly contemporaneously as events are happening to the patient. For example, a “real-time clinical evaluation” can evaluate how medication administration(s) (e.g., types, amount, dosage, at what time of day) affect a patient throughout the day, at different times during the day, and/or when a relevant task is being performed by the patient.

A “computer” is a programmable machine designed to sequentially and automatically carry out a sequence of arithmetic or logical operations or steps. The particular sequence of operations or steps can be changed readily, allowing the computer to solve more than one kind of problem. The term “computer” includes reference to a machine that performs calculations automatically. A “computer” may be large or small, as well as mobile (such as a mobile device) or otherwise. For example, the term “computer” encompasses a mobile computer device such as a smart phone or tablet.

The term “mobile device” or “mobile computer device” (also known as a cellphone device, handheld device, handheld computer, tablet computer, net book, laptop or smart phone) refers to a portable electronic device typically having a display screen with touch input and/or a miniature keyboard which processes, receives and sends data without the need to maintain a wired connection with the internet. Examples of mobile devices include, but are not limited to, iPhone®, iPad®, Blackberry®, Android®, etc. The term “smart phone” refers to an electronic handheld device that integrates the functionality of a mobile phone, personal digital assistant (PDA) or other information appliance. The term “tablet computer” refers to a slate—or tablet—shaped mobile computer device, equipped with a touchscreen or stylus. The present invention contemplates relevant applications or “APPs”, for example in a Mac or PC format, that run on a mobile computer device via typing, a touchscreen or touch pad via stylus or manual touch.

The term “calculating” means to carry out a sequence of arithmetic or logical operations or steps for the purpose of generating or providing a result. The term “providing” means to gather, show, send, transmit, transfer, transform or create information or results, for example via one or more computers.

The term “database” refers to a machine and specific data structure for collecting, structuring and organizing and analyzing data in electronic format and the data collected from a digital device that is so structured and organized. In certain embodiments, this structure accounts for each of the major variables defined above, such as: medication type, dose and time of administration, personality characteristics, task descriptions, characteristics, complexity and familiarity, as well as the patients familiarity and interest in the task, environmental characteristics such as external distractions and interruptions, and internal motivational and reward factors. In certain embodiments, other relevant factors include independent-of-medication variables (intrinsic factors), such as the patient's energy level and vitality on a specific day, which might be effected by the patient's quality of sleep on the previous night, or the presence of a headache or stomachache, etc, on the day of the task.

The term “graphical user interface (GUI)” refers to a type of software program that facilitates interaction between a user and a computing device.

The term “receiving information” refers to a process in which a device, system, computer and/or software acquires, or is given or presented with, knowledge, facts, data, or information. In one aspect of the present invention, a relevant interface is intuitive, simple, readable, and rapidly enterable.

The term “clinical evaluation” refers to the appraisal of the data, such as data generated by use of a digital, computer and/or mobile computer device, that gives rise to the diagnosis and/or treatment of patients. The phrase “results indicative of the effects experienced” by a patient upon taking medication may include a summary, chart, table, analysis, transmission and/or presentation of information, and/or a recommendation, prediction and/or warning, regarding current and/or future treatment and administration of medication

The term “medication” refers to a drug, biologic or device intended for use in the medical

The term “disorder” refers to a physical and/or mental condition in which there is a disturbance of normal functioning.

The term “task” refers to an activity or piece of work that is undertaken or attempted by a subject or patient.

The term “environment” refers to aspects of the area in which a patient exists and the totality of that areas' surrounding conditions.

The term “rating” refers to an evaluation or appraisal of the degree to which something affects a subject (e.g., impairs, enhances, improves, worsens or has no impact), or is desirable or undesirable to the subject. For example, a “severity rating” may refer to evaluating or appraising the degree to which something is undesirable or otherwise impacts a mental or physical state, symptom, feeling or well being of a patient. The phrases “impact on functioning rating” or “functioning rating” refer to evaluating or appraising the degree to which something affects how a person operates or works on one or more various tasks. One can “rate” a treatment protocol or effect or impact of medication on a patient (i.e., “rating” can be measured, determined, summarized, presented or documented) in many different ways, such as in terms of: (a) none, mild, moderate, severe and/or extreme, (b) poor, low, good, superior and/or excellent, (c) worse, none, some, great and/or extreme, (d) not improved and/or much improved; (e) none or a lot, and/or (f) on a scale, such as a number scale (e.g., on a scale from 1-2, 1-3, 1-4, 1-5, 1-6, 1-7, 1-8, 1-9 or 1-10).

The term “treatment recommendation” refers to, for example, a preferred/advisable medical procedure or application, medication, dosage and time of administration for a patient based on known patient information, including information obtained based on a real-time evaluation of a patient, as well as previously gathered information about the patient and existing treatment regiment.

The term “psychiatric disorder” refers to any neurobehavioral or psychological state of a patient that impacts the patient's well being, physical and mental health, state of mind, and ability to perform tasks. For example, a “psychiatric disorder” may include an attention-deficit hyperactivity disorder (e.g., ADHD and/or ADD), an anxiety disorder (e.g., obsessive compulsive disorder, OCD), a mood disorder (e.g., depression or bipolar), a thought disorder (e.g., Aspergers) or substance abuse. A “psychiatric disorder” may also include hyperactivity, impulsiveness, an oppositional defiant disorder (ODD), over-reactive emotion and a learning disability.

An “anxiety disorder” refers to a mental disorder in which severe anxiety is a salient symptom. A mood disorder refers to a disorder in which a disturbance in a person's mood is a salient symptom. A thought disorder refers to a pattern of disordered language use that is presumed to reflect disordered thinking and may be considered a symptom of psychotic mental illness. Substance abuse refers to the overindulgence in and dependence upon a drug or other chemical leading to effects that are detrimental to the individual's physical and mental health, or the welfare of others. Obsessive compulsive disorder (OCD) refers to an anxiety disorder characterized by intrusive thoughts that produce uneasiness, apprehension, fear, or worry, by repetitive behaviors aimed at reducing the associated anxiety, or by a combination of such obsessions and compulsions. Depression refers to mental state characterized by a pessimistic sense of inadequacy and a despondent lack of activity. Bipolar refers to a mental illness characterized by two opposite and extreme types of moods: episodes of mania (hyperactivity, excessive cheerfulness and excitement, decreased need of sleep, flight of ideas, etc.) and depression (marked by poor appetite and poor self-esteem, sleep disturbances (e.g., insomnia or oversleeping), hopelessness, loss of energy, suicidal ideas, etc.). Asperger refers to a disorder that is characterized by significant difficulties in social interaction, along with restricted and repetitive patterns of behavior and interests.

The term “hyperactive” refers to a physical state in which a person is abnormally and easily excitable or exuberant. The term “impulsive” refers to characterized by undue haste and lack of thought or deliberation. The term “oppositional defiant disorder (ODD)” refers to an ongoing pattern of uncooperative, defiant, and hostile behavior toward authority figures that seriously interferes with the person's day-to-day functioning. The term “over-reactive emotion” refers to a disorder characterized by a person being easily upset, frustrated, hard or slow to calm down, often or easily angered, overly self critical, and overly critical of others. The term “learning disability” refers to a disorder found in children of normal intelligence who have difficulties in learning specific skills.

Following below are more detailed descriptions of various concepts related to, and embodiments of, inventive systems, methods and apparatus for real-time clinical evaluation. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Applicant has given considerable attention to the analytic process required to integrate the factors discussed herein. The fundamental underlying presumption reflects awareness that not all factors (e.g. diagnoses, tasks, personality, environment, etc) are equivalently weighted or relevant. While some presumptions are made in the initial analysis of preliminary data, the ultimate formula for weighing relative significance will be empirically derived by repeated measures and refinement in interest of determining a Best Fit. Multiple putative factors are repeatedly retrospectively reanalyzed to refine predictability by continuously re-weighing individual factors, and discarding or adding unanticipated ones. Applicant's modeling and analysis is subject to fluid refinement as the empirical data warrants. It accommodates the addition of new medications as they emerge and optimizes the relative impact of associated disorders. Applicant recognizes that the significance of a diagnosis is not uniform. A little ADD or a modest degree of OCD or anxiety can be a functional advantage allowing increased vigilance or greater caution. A higher degree of the same symptoms can become significantly dysfunctional, leading to a lack of depth of focus, or an inability to shift attention appropriately, or inappropriate avoidance of a task that seems difficult. In certain embodiments, the present invention provides specific rating scales for measuring these and other variables with preliminary data suggestive of cut-offs or dimensions for increasing dysfunction. Applicant has created an analytic model that accounts for these variables, but allows progressive refinement of their significance.

### Overview

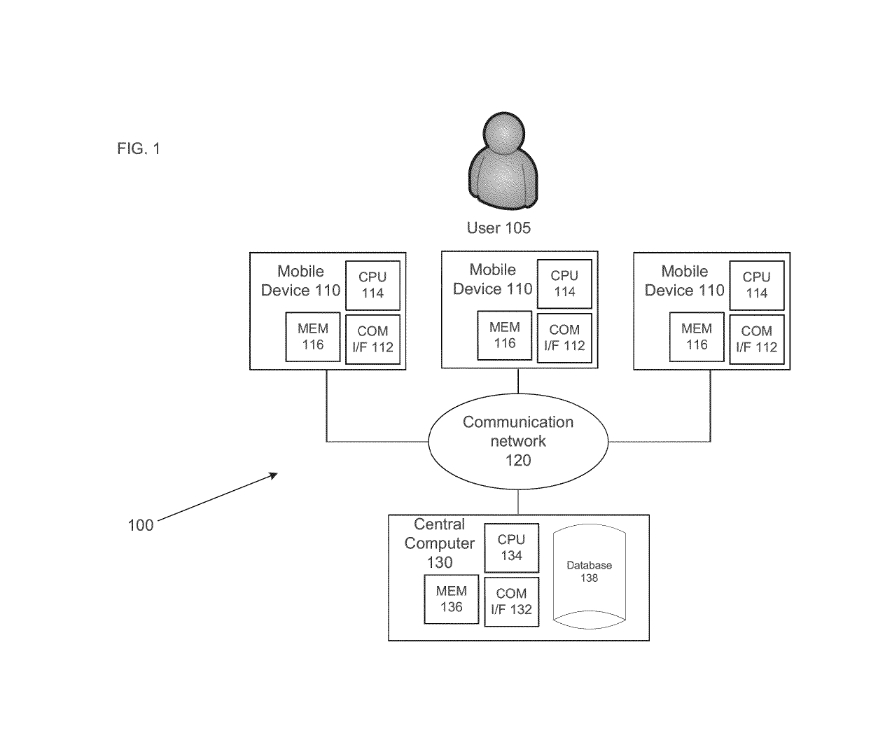

FIG. 1 illustrates a functional block diagram of a real-time clinical evaluation system 100, according to one embodiment of the present invention, for identifying one or more effects (such as a benefit, lack of benefit, and/or side effect) of a medication for the treatment of a disorder in a patient.

In various aspects, the real-time clinical evaluation system 100 includes one or more instrumentation components, computer programs, software (i.e., non-transitory computer-readable medium) and/or computing devices (e.g., one or more computers, portable/handheld computing devices, etc.), and utilizes a communications infrastructure (which at least in part may employ network elements and/or dedicated communication links, components, devices, etc.) to provide communication of information amongst respective components/devices of the system and various parties from which relevant information may be acquired and/or to which information may be provided.

While FIG. 1 illustrates a number of system components/devices and parties that may exchange information with each other as part of implementing the real-time clinical evaluation system 100, it should be appreciated that not all of the components/devices/parties shown in FIG. 1 are necessarily required to implement the various embodiments discussed herein of real-time clinical evaluation systems, and associated methods and apparatus. In particular, in various embodiments, some or all of the components/devices shown in FIG. 1, in a variety of combinations, may be employed to realize a particular implementation of real-time clinical evaluation system 100 according to the present invention.

Various implementations of the systems and techniques described herein can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications, applications, apps or codes) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

In general, as shown in FIG. 1, the real-time clinical evaluation system 100 may include, but is not limited to, a mobile device 110, a communication network 120 and a central computer 130.

Communication network 120 provides for communication between two or more components/devices relating to the real-time clinical evaluation system 100. For example, in one embodiment, network 120 provides the communication infrastructure by which information may be exchanged between any one or more of the mobile device 110 and central computer 130. Communication network 120 may be, for example, any local area network (LAN) and/or wide area network (WAN) for connecting to the Internet. Further, the communication network 102 may be a cellular communications network. In addition, the network may facilitate radio communication such as two-way radio transmission. Moreover, communication network 120 may be comprised of one or more different communications networks that may be of different types. The system described herein may be implemented using “cloud computing,” which encompasses off-site or central storage of large data sets for complex, multi-factor analysis. Regardless of the implementation, the system is intended to provide the highest level of privacy and security required for sensitive personal medical data.

As shown in FIG. 1, respective components/devices of the real-time clinical evaluation system 100 may include one or more communication interfaces to facilitate communication of various information. For example, a communication interface 112 of mobile device 110, and a communication interface 132 of central computer 130 may be employed to provide connectivity to other components/devices of the system 100 (e.g., via network 120).

Communication interfaces 112, and 132 may be any wired and/or wireless communication interfaces by which information may be exchanged between any components/devices of the real-time clinical evaluation system 100. Example wired communication interfaces may include, but are not limited to, USB ports, RS232 connectors, RJ45 connectors, Ethernet, and any combinations thereof. Example wireless communication interfaces may include, but are not limited to, Bluetooth technology, Wi-Fi, Wi-Max, IEEE 802.11 technology, radio frequency (RF), LAN, WAN, Internet, shared wireless access protocol (SWAP), Infrared Data Association (IrDA) compatible protocols and other types of wireless networking protocols, and any combinations thereof. The present invention recognizes the added burden of confidentiality and privacy that must attend any use of wireless data transmission and are generating multilevel (e.g., 2 levels, 3 levels, etc.) of password protection for such transmissions.

As also shown in FIG. 1, one or more components/devices of clinical evaluation system 100 generally includes a memory (e.g., one or more computer-readable storage media) to store processor-executable instructions as well as other data (e.g., see memory 116 and 136). One or more components/devices also may include one or more processing units (e.g., a microprocessor, microcontroller, FPGA, etc.; see processing units 114 and 134) communicatively coupled to the communication interface and the memory, wherein upon execution of the processor-executable instructions by the processing unit, the processing unit performs a variety of functions as set forth in greater detail below for respective components/devices. Generally speaking, many of the functionalities described herein and attributed to various components/devices of or in communication with the clinical evaluation system 100 shown in FIG. 1 may be encoded as processor-executable instructions stored in/on one or more computer-readable storage media.

As shown in FIG. 1, the central computer may include a database 138 to organize, store and retrieve large amounts of data. The database 138 may include bibliographic, document-text, and statistical data. In addition, database 138 allows for data creation, maintenance, search and other access.

User 105 uses the clinical evaluation system 100. User 105 may be a patient, parent/guardian of the patient, teacher, parent tutor, mentor, coach or other interested party associated with the patient. Preferably, user 105 operates mobile device 110. Specifications of mobile device 110 and central computer 130 will be described in detail later in this application. Having described certain components of various embodiments of a real-time clinical evaluation system 100 of the present invention, additional details of the various methods performed by real-time clinical evaluation system 100 are now provided. Patients will be able to score their performance and medication effectiveness as evident and recalled over the past week and perhaps past month. However, they will also score an abbreviated index of medication effectiveness in real time on a hourly or every other hour basis. The larger time-frame provides an overall index of medication effectiveness and tolerability. The hourly scoring provides a sensitive measure of the duration of medication effect and the specificity of that effect to the type of actual task being performed. Both forms of information are relevant, but the hourly measures are most unique metabolically and are task-specific.

### Real-Time Clinical Evaluation

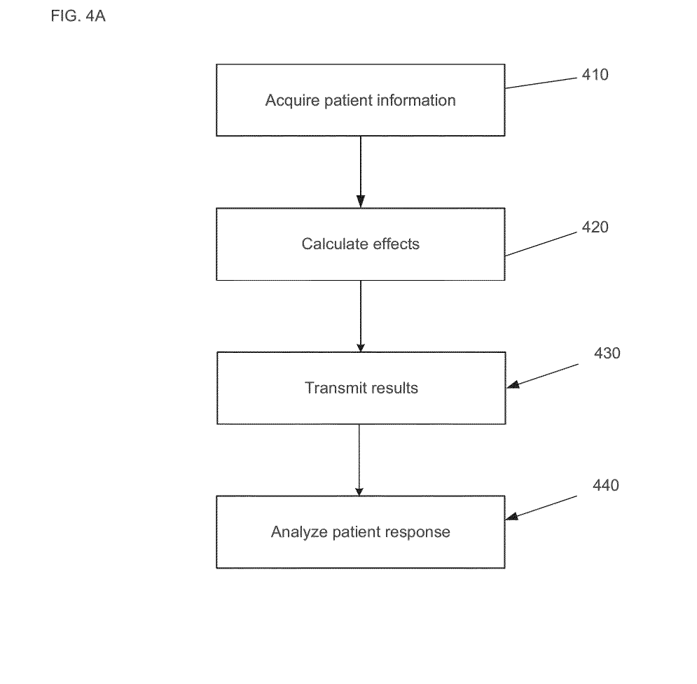

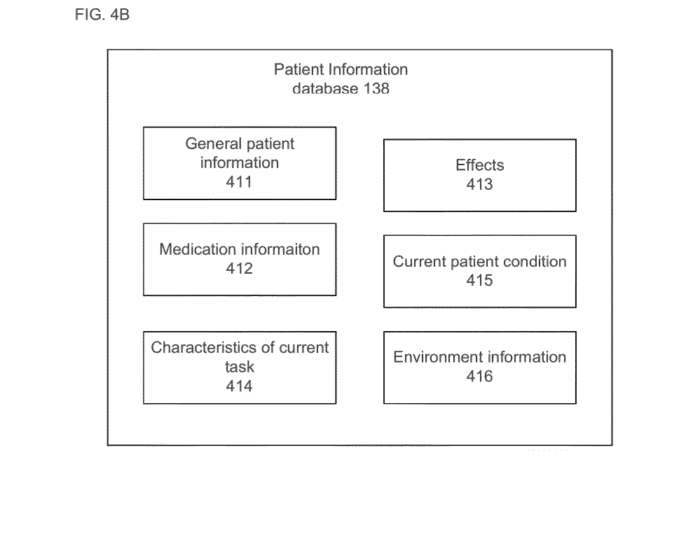

FIG. 4A provides a flow chart to graphically illustrate a method for real-time clinical evaluation of one or more effects (e.g., benefit, lack of benefit, or side effect) of a medication for the treatment of a disorder in a patient (various functionalities of which may be generally implemented by the real-time clinical evaluation 100 of FIG. 1), according to embodiments of the present invention.

The disorders being treated may include one or more selected from a group consisting of ADD and/or ADHD, an anxiety disorder, a mood disorder, a thought disorder or substance abuse. Most patients treated in psychiatric practice (as opposed to pediatric practice) have more than one diagnosis. Typical ADD patients often have associated depression, demoralization, and may have primary or secondary anxiety, and some degree of alcohol or substance abuse. The ADHD may be selected from the group consisting of ADHD—Predominately Hyperactive-Impulsive Type; ADHD—Predominately Inattentive Type (ADD); and ADHD—Combined Type. Further, the ADHD may be adult ADHD or child ADHD. The anxiety disorder may be obsessive-compulsive disorder (OCD). The mood disorder may be depression, demoralization, and/or bipolar disorder. The thought disorder may be Aspergers or overt psychosis. Aspergers reflects a significant disturbance in empathy and social connection. Thought disorder implies a disruption in the meaning or connection of words or gross misattributions of causation. This may be attributed to distorted religious or “scientific” beliefs, or a longing for a magical solution to pain, isolation, emotional emptiness, or threatening thoughts of inappropriate punishment for imagined acts. The substance abuse may involve previous or current abuse by the patient of a substance selected from the group consisting of alcohol, amphetamines, barbiturates, benzodiazepines, cocaine, methaqualone, opioids or synthetic hallucinogens.

The medication being taken by the patient may be a psychostimulant. Although psychostimulants share the common benefit of enhancing attention, these medications differ widely in both their onset and duration of action (pharmacokinetics) and in their impact on task performance. For example, generally methylphenidates (MPHs), such as Ritalin and Concerta, are more inhibitory. They enhance quieting of behavior and doing one task at a time. Amphetamines (AMPHs), such as Adderall and Vyvanse, enhance energy and facilitate wider, more flexible attention. Even within AMPHs, there are clinical differences in impact on attention. For instance, Vyvanse enhances wider more flexible attention compared to Adderall that increases depth of narrowly focused attention. In addition, Vyvanse increases the ability to shift from one task to another provided the tasks are familiar and do not require great learning. Vyvanse also tends to last longer (e.g., 10-12 hours) as compared to Adderall (e.g., extra-strength 8-10 hours; regular strength 3-5 hours). Adderall enhances depth of attention essential for study and learning. For example, many college students and working executives may want to take a morning dose of Vyvanse, which enhances their general attention throughout the day. For periods when they need to study or learn new material, they may want to take a pulse dose (a single dose timed to the task) of regular Adderall to enhance attention for learning or writing detailed reports.

Effects of medication on patients are clearly impacted by tasks to be performed by the patient, the medication itself, dosages and timing of dosages, all of which underlie the broad concept of a “Best Fit” and are quantifiable using the real-time clinical evaluation system 100. The real-time clinical evaluation system 100 allows patients to record their objective and selective impressions of a task they are performing. The real-time clinical evaluation system 100 takes into account both (a) characteristics of the task (e.g., multitasking, complexity, urgency, risk, importance, abstract, concrete, cognitive, motor, organization, presentation, creation, production, etc.) and (b) patient factors (e.g., interest, familiarity, learning required, motivation, etc.). The effect of medication on task performance can be measured and tracked in real-time, e.g., immediately before, soon after or concurrently with a task being performed. Accordingly, the effects of medication as it relates to task characteristics and performance may be used by real-time clinical evaluation system 100 to determine a “Best Fit” medication for a patient in relation to the tasks they perform at specific times throughout the day.

In the method outlined in FIG. 4A, at block 410, real-time patient information is acquired by real-time clinical evaluation system 100. For example, with reference to FIG. 1, a central computer 130 receives the patient information. Central computer 130 may be a personal computer, workstation, server, etc. The patient information may have been input directly into central computer 130 or transmitted from another device such as mobile device 110. The mobile device 110 may periodically prompt a user 105 to enter patient information into the mobile device 110 and transmit the patient information to the central computer 130. For example, the mobile device 110 may prompt the user 105 to enter information at least daily or at least once a week.

Central computer 130 may also contain a database 138 that is configured to store the real-time acquired information. Prior to the reception of the real-time patient information, other patient information (e.g., patient history) may also be stored in database 138. The information stored in the database 138 can be stored in compliance with the Health Insurance Portability and Accountability Act (“HIPAA”), and the patient data-privacy and confidentiality regulations promulgated thereunder. The information stored in the database 138 allows for the integration of empirical measures of personality, neurobiology and task specificity to determine medication selection, dosing and timing. Further, medication selection, dosing and timing may be tested and implemented through application of the comprehensive database 138 that may relate each domain of information to every-other treatment variable. This database system provides the framework for connecting causes of psychiatric disorders (e.g., environmental, traumatic, emotional, neurobiological, genetic, etc.) to each other. Not only does the database system help define the selective contribution of the precipitants, but it also helps identify the relative value of selected treatments. In addition, the database 138 may become a holding mechanism for integrating multiple domains of clinical and brain functional information into specific treatment efficacy decision-making. Further, the database 138 may provide the framework for clinical validation of new assessment methods.

FIG. 4B illustrates a block diagram of acquired patient information. As indicated above, patient information includes at least two types of information. The first type of patient information that is stored in the database is general patient information 411. This information may include the patient's medical history, prior symptoms, developmental history, ability to metabolize the medication (pharmacokinetics), receptor sensitivity to given blood levels of the medication (pharmacodynamics), personality, temperament, results from brain imaging, computer Q-EEG information, results of medical, diagnostic or genetic testing, quality of life, use of prescription, over-the-counter or illegal drugs, hormone levels, etc. This stored information may also include information relating to specific characteristics of the medication, such as what isomer is used, duration in the body (long versus short acting variants), and whether a medication is in generic form, which can provide up to 20% variability in dose from the branded version. Such characteristics can impacting dose, rate of release, absorption into the blood stream, excretion half life and rate of disappearance from circulation. A medical professional in conjunction with the real-time clinical evaluation system 100 may use such information to help refine clinical decision-making in relation to other diagnostic and neurological measures. In certain embodiments, the present invention includes training materials, as DVDs, web casts, written and published documents, etc., to assist physicians in the interpretive process and to develop empirical consensual models for interpretation and application within the limits of the data's useful validity.

The second type of patient information is the additional or real-time patient information 412-416 received by database 138 for use in conducting a real-time clinical evaluation. The patient information may include at least one of a medication being taken by a patient for the treatment of a disorder 412, the effects 413 of the medication on the patient, characteristics of a current task being performed by the patient 414, a current functioning baseline of the patient 415 and environmental information 416.

In certain embodiments, the information concerning the medication being taken by the patient 412 comprises at least one selected from the group consisting of a name or names of medication, frequency of administration of the medication, dosage of the medication, the time at which the patient takes the medication, and the different medication's method of release. The information concerning the effects 413 of the medication to the patient is a rating of at least one of the patient's energy, interest, focus, concentration, organization, prioritization, productivity, efficiency, quality of work and work product.

The information concerning characteristics of a current task being performed by the patient 414, comprises an objective description of the task being performed by the patient, a patient's subjective (as perceived by the patients themselves) description of the task being performed by the patient and information relating to a setting in which the task is being performed. The objective description of the task being performed by the patient may be related to at least one of urgency, motor, cognitive, a type of thinking required, learning, memorization, presenting, creativity, complexity, variability and importance. The subjective description of the task being performed by the patient may be related to at least one of familiarity, whether the task requires new learning, interest, enjoyment, effort, energy and relaxation. The setting in which the task is being performed may be at least one of home, work, recreation and in transit, and indicate degree of support and/or degree of distraction.

The information concerning a current condition of the patient 415 may include information concerning how the patient is feeling, information concerning the patient's inattention, information concerning the patient's hyperactivity, information concerning the patient's impulsivity, information concerning experiences of the patient in a setting and information concerning side effects being experienced by the patient. The information concerning how the patient is feeling may be related to at least one of mood, energy level, alertness, motivation, attention, frustration, anxiety, whether the patient is worried and whether the patient is obsessive. The information concerning the patient's inattention may be related to at least one of distraction, concentration, attention, carelessness, whether the patient misplaces items, forgetfulness, prioritization, organization, whether the patient avoids complex tasks and whether the patient finishes tasks. The information concerning the patient's hyperactivity may be related to at least one of activity, whether the patient is fidgety and restlessness. The information concerning the patient's impulsivity may be related to at least one of whether the patient has difficulty waiting their turn, whether the patient has difficulty relaxing, whether the patient talks too much, whether the patient finishes others sentences and whether the patient interrupts others.

The information concerning experiences of the patient in an environment or setting 416 may be related to at least one of expectations, distractions, interruptions, demands, tension, schedule changes, supportiveness, resources and assistance. Specifically, in some environments there are a lot of external distractions or many interruptions that impair or disrupt the flow of attention. Some of these distractions and frustrations can be quite concrete, e.g., not having the supplies needed, the printer or computer doesn't work. There are also emotional or affective domains to tasks which may impact the general emotional tone of the environment. Business and Human Resource publications elaborate these dimensions in depth in articles regarding “what constitutes an effective and motivating work environment.” These environmental factors also link to the personality characteristics of employees, as well as their specific work skills. Some employees require great clarity of task and mission to be effective: they want to learn and know a limited skill-set and the do routine tasks that utilize what they know. More innovative companies thrive on invention and change. Promotion and advancement in such an environment requires contribution to innovation and linked to production and marketing.

At block 420 of FIG. 4A, results indicative of effects (e.g., benefits, lack of benefits, side effects, etc.) experienced by the patient upon taking the medication are calculated or provided based on the received information. The results indicative of benefits to the patient may relate to at least one of energy, focus, organization, productivity and quality of life of the patient. The results indicative of side effects experienced by the patient relates to at least one of appetite, insomnia, headache, stomachache, irritability, sedation and fatigue.

The calculation in block 420 may be performed several ways. In one configuration, the calculation is performed by software running on the mobile device 110. In another example, information collected at mobile device 110 is transferred to central computer 130 via communications network 120 and the calculation is performed by software running on the computer 130. In certain embodiments, the software applies an algorithm on the stored patient information and the acquired real-time current information to obtain an evaluation result. Alternatively, medical personnel (e.g., doctor, nurse, physician assistant) may be presented with the stored patient information and real-time current information, and make a determination of effects of the medication based on this information and medical expertise. In certain embodiments, software or medical personnel calculates, determines, recommends, or provides information relating to alterations or changes in medication, dosage, or delivery mechanism that may benefit or otherwise impact the patient, or increase the patient's likelihood of maintaining the current medication regimen.

At block 430, the information calculated or provided at block 420 is transmitted to an interested party. According to one embodiment, the results are transmitted from central computer 138 to mobile device 110 so that they may be communicated (audibly, visually or both) to user 105.

At block 440, the patient's response to the medication is analyzed based on the received information and patient information that was previously stored in the database 138. The evaluation at block 440 may be performed several ways. In one example, the evaluation is performed by software running on the computer 130. The software applies an algorithm on the stored patient information and the received real-time current information to obtain an evaluation result. Alternatively, medical personnel (e.g., doctor, nurse, physician assistant) are presented with the stored patient information and the received real-time current information via the central computer 130 and make a determination of the effectiveness of the medication based on this information and their own clinical experience and medical expertise. This analysis can then be used to modify and/or confirm the use of the medication and that recommendation can be shared with the patient.

The patient's response calculation may make use of several techniques including, but not limited to, analytics, statistical methods and Bayesian statistics. The analytics integrate and weigh the relative importance/significance of personality, diagnostic, and neurobiological measures to selection of specific medication and measures of treatment effectiveness. These analytics will help determine whether the patient is on the right medication at the optimal dose and time for the specific tasks he is performing Statistical methods will be used to analyze patient and physician data to determine the contrition of each major variable to the “Best Fit” results of medication effectiveness. In addition, regression analysis may identify patterns of effects—such as the contribution of personality factors or of neurobiological/genetic variables to the medication response. Finally, Bayesian statistics may be utilized to determine the combined effect of multiple simultaneous variables on a specific result—such as optimal medication, dosage and delivery mechanism selection.

Accordingly, information concerning a patient on medication is collected. In one embodiment, because the information is collected via a mobile device 110, the information can be collected in real-time. That is, the patient can enter information as he experiences various conditions instead of having to rely on memory. This real-time information can be analyzed to determine the effects of the medication. Further, the real-time information can be used to analyze the patient's response to the medication. This analysis can then be used to modify and/or confirm the use of the medication and that recommendation can be shared with the patient.

Having described generally a real-time clinical evaluation method according to various embodiments of the present invention, additional details of the method performed by real-time clinical evaluation system 100 are now provided.

### Optimization of the Effectiveness of a Medication for the Treatment of a Disorder

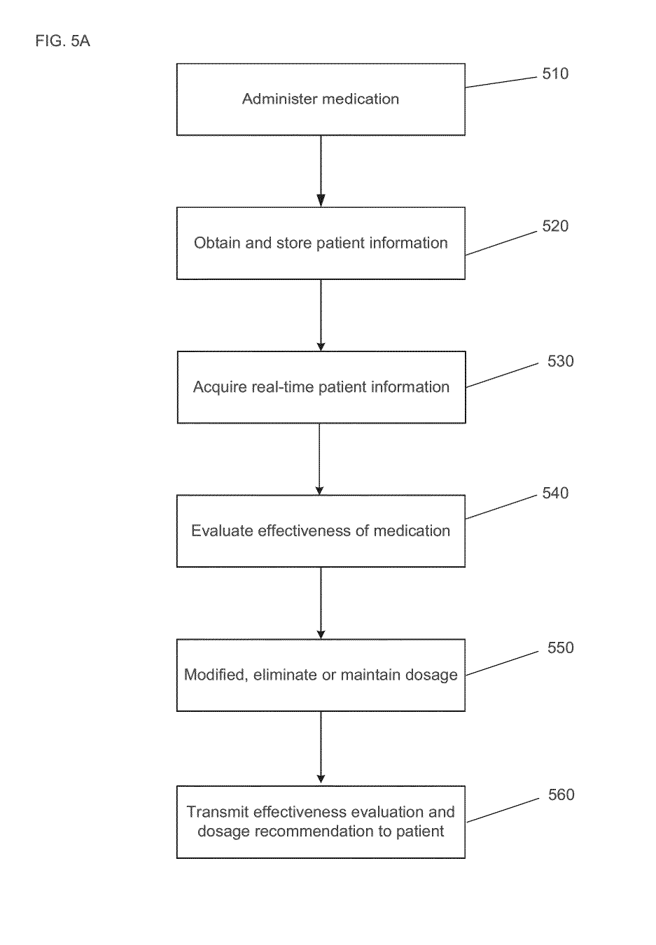

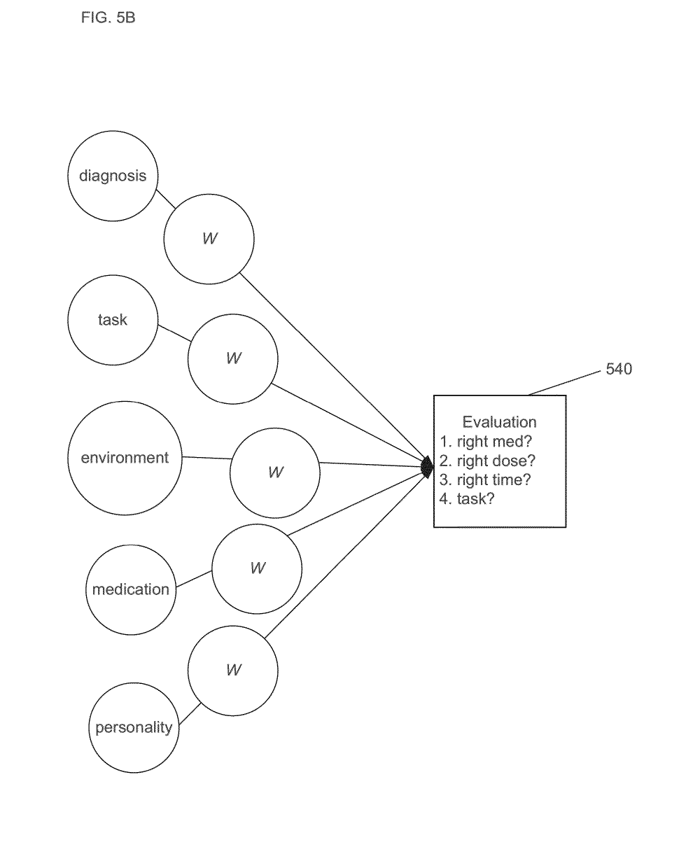

FIG. 5A provides a flow chart to graphically illustrate a method for optimizing the effectiveness of a medication for the treatment of a neurobehavioral or psychiatric disorder in a patient (various functionalities of which may be generally implemented by the real-time clinical evaluation 100 of FIG. 1), according to embodiments of the present invention. The neurobehavioral or psychiatric disorder may be selected, for example, from the group consisting of an attention-deficit hyperactivity disorder (ADHD), an anxiety disorder, a mood disorder, a thought disorder or substance abuse. The ADHD may be selected from the group consisting of ADHD—Predominately Hyperactive-Impulsive Type; ADHD—Predominately Inattentive Type (ADD); and ADHD—Combined Type. Further, the ADHD may be adult ADHD or child ADHD. The anxiety disorder may be obsessive-compulsive disorder (OCD). The mood disorder may be depression and/or bipolar disorder. The thought disorder may be Aspergers. The substance abuse may involve previous or current abuse by the patient of a substance selected from the group consisting of alcohol, amphetamines, barbiturates, benzodiazepines, cocaine, methaqualone and opioids, hallucinogens and emerging synthetic agents (e.g., ecstasy, GHB, etc.).

In the method outlined in FIG. 5A, at block 510, a medication is administered (at a predetermined dosage and frequency) to a patient for the treatment of a neurobehavioral or psychiatric disorder. The medication may be administered to the patient over the course of a predetermined trial period (e.g., two weeks) during which the patient provides real-time information concerning their condition via the real-time clinical evaluation system 100.

At block 520, with reference to FIG. 4B, general information about the patient 411 is obtained and stored in a central computer database 138. The patient information may relate, for example, to at least one selected from the group consisting of the patient's medical history, prior symptoms, developmental history, ability to metabolize the medication (pharmacokinetics), receptor sensitivity to given blood levels of the medication (pharmacodynamics), personality, temperament, one or more current tasks, results from brain imaging, computer Q-EEG information, results of medical, diagnostic or genetic testing, quality of life, use of prescription, over-the-counter or illegal drugs, and hormone levels.

At block 530, real-time clinical evaluation system 100 acquires real-time patient information. For example, with reference to FIG. 4B, a user 105 (the patient or person associated with the patient) having a mobile device 110, inputs and sends in real-time current information that may relate to the medication being taken by the patient 412, at least one effect 413 of the medication on the patient, characteristics of a current task being performed by the patient 414, a current condition of the patient 415 and the environment/setting of the patient 416. The mobile device 110 may periodically prompt a user 105 to enter patient information into the mobile device and transmit the patient information to the central computer 130. For example, the mobile device 110 may prompt the user 105 to enter information at least daily or at least once a week.

The information concerning the medication being taken by the patient 412 comprises, for example, at least one selected from the group consisting of a name or names of medication, frequency of administration of the medication, dosage of the medication, the time at which the patient takes the medication, and the method of release of the medication. The information concerning the effects 413 of the medication to the patient may be a rating of selected factors related to the patient's energy, interest, focus, concentration, organization, prioritization, productivity, efficiency, quality of work and work product.

The information concerning characteristics of a current task being performed by the patient 414, comprises an objective description of the task being performed by the patient, a patient's subjective (as perceived by the patient themselves) description of the task being performed by the patient and information relating to a setting in which the task is being performed. The objective description of the task being performed by the patient may be related to selected factors related to urgency, motor, cognitive, a type of thinking required, learning, memorization, presenting, creativity, complexity, variability and importance. The subjective description of the task being performed by the patient may be related to domains such as familiarity, whether the task requires new learning, interest, enjoyment, effort, energy and relaxation. The setting in which the task is being performed may be at least one of home, work, recreation and in transit, and indications of the degree of support and/or degree of distraction.

The information concerning a current condition of the patient 415 may include information concerning how the patient is feeling, information concerning the patient's inattention, information concerning the patient's hyperactivity, information concerning the patient's impulsivity, information concerning experiences of the patient in a setting and information concerning side effects being experienced by the patient. The information concerning how the patient is feeling may be related to selected factors of mood, energy level, alertness, motivation, attention, frustration, anxiety, whether the patient is worried, whether the patient does not feel like him/herself, and whether the patient is obsessive. The information concerning the patient's inattention may be related to at least one of distraction, concentration, attention, carelessness, whether the patient misplaces items, forgetfulness, prioritization, organization, whether the patient avoids complex tasks and whether the patient finishes tasks. The information concerning the patient's hyperactivity may be related to at least one of activity, whether the patient is fidgety and restlessness. The information concerning the patient's impulsivity may be related to at least one of whether the patient has difficulty waiting their turn, whether the patient has difficulty relaxing, whether the patient talks too much, whether the patient finishes others sentences and whether the patient interrupts others.

The information concerning experiences of the patient in an environment or setting 416 may be related to relevant domains of expectations, distractions, interruptions, demands, tension, schedule changes, supportiveness, resources and assistance. Specifically, in some environments there are a lot of external distractions or many interruptions that impair or disrupt the flow of attention. Some of these distractions and frustrations can be quite concrete, e.g. not having the supplies needed, the printer or computer doesn't work. There are also emotional or affective domains to tasks which may impact the general emotional tone of the environment. Business and Human Resource publications elaborate these dimensions in depth in articles regarding “what constitutes an effective and motivating work environment.” These environmental factors also link to the personality characteristics of employees, as well as their specific work skills. Some employees require great clarity of task and mission to be effective: they want to learn and know a limited skill-set and how to do routine tasks that utilize what they know. More innovative companies thrive on invention and change. Promotion and advancement in such an environment requires contribution to innovation and is often linked to production and marketing.

At block 540, the effectiveness of the medication for the treatment of the neurobehavioral or psychiatric disorder in the patient is evaluated based on stored patient information and received real-time current information. The evaluation of block 540 may be performed several ways. In one example, the evaluation is performed by software running on the mobile device 110 and/or computer 130. For example, the software applies an algorithm on the stored patient information and the received real-time current information to obtain an evaluation result. Additionally, medical personnel (e.g., doctor, nurse, physician assistant) may be presented with the stored patient information and the received real-time current information via the central computer 130 and make a determination of the effectiveness of the medication based on the trends noted in this information and medical expertise.

In certain embodiments, the underlying concept of an effective “Best Fit” prediction depends on two basic ideas: (1) the identification of key factors and (2) the relative weighting or contribution of these components. For example, for a patient suffering from ADD/ADHD, key factors include: (a) Diagnoses: the relative functional impact of each diagnosis (Dx), (b) Personality: characterized in three axes of Novelty Seeking (NS), Harm Avoidance (HA), and Reward Dependence (RD), (c) Tasks: the attentional demand of the essential tasks performed—both Objective and Subjective characteristics, (d) Environment: distractions, motivational reinforcements and subjective impact and (e) Medications: effects on wide and narrower attention, the duration of effect, patient comfort and preference with the prescribed meds.

In one embodiment, and as shown in FIG. 5B, an algorithm for determining a qualitative “Best Fit” value is:

Best Fit=(Diagnosis)W×(Medication(Type,Dose,Time))W×(Task-Objective)W×(Task-Subjective)W×(Personality)W×(Environment)W.

Each variable may be weighted by a value W based on various predetermined settings of the real-time clinical evaluation system 100.

In certain embodiments, factors relevant to “best fit” include diagnoses, relevant impact of each diagnosis on functioning, nature of actual tasks performed, medication (e.g., type, dose, time), patient ability to metabolize medication (pharmacokinetics), patient receptor sensitivity to given blood levels of medication (pharmacodynamics), timing of tasks in relation to medication, the setting (environment in terms of distractions, reward and reinforcement), personality factors, including sensitivity to internal versus external rewards, and other factors.

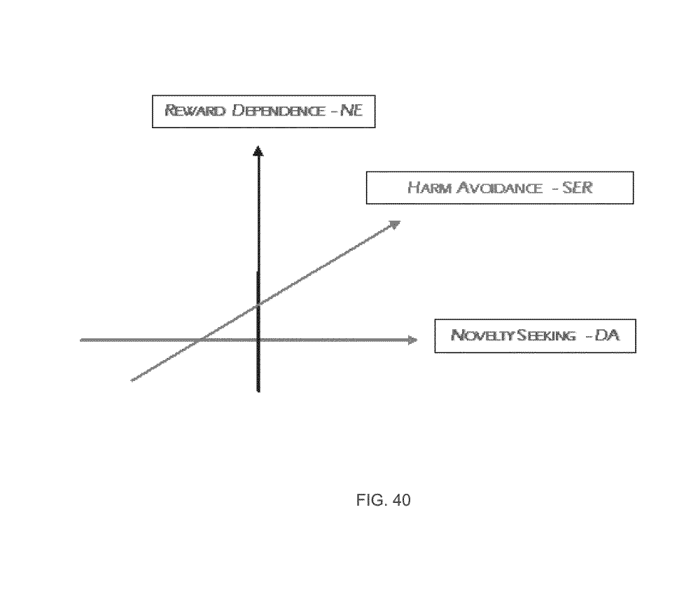

In one embodiment, personality factors are defined in terms of three axes that reflect neurobiological factors that are relevant to pharmacological treatment. FIG. 40 illustrates the three axis, i.e., novelty seeking (NS), harm avoidance (HA) and reward dependence (RD), which are regulated principally by monoamine neuromodulators dopamine (DA), serotonin (SER) and norepinephrine (NE), respectively, in brain systems.

Novelty seeking (NS) refers to a tendency toward frequent exploratory activity and intense exhilaration in response to novel or appetitive stimuli. High novelty seeking can result in impulsive, exploratory, fickle, excitable, quick-tempered, extravagant, and disorderly behaviors. Low novelty seeking can result in reflective, rigid, loyal, stoic, slow-tempered, orderly, and persistent behaviors.

Harm avoidance (HA) can refer to a tendency to respond intensely to aversive stimuli and their conditioned signals, thereby facilitating learning to inhibit behavior in order to avoid punishment, novelty, and frustrative omission of expected rewards. Harm avoidance can result from a behavioral inhibition brain system that regulates passive avoidance and extinction responses to conditioned signals of punishment, novelty or frustrative nonreward. Patients with high harm avoidance are often cautious, tense, apprehensive, fearful, inhibited, shy, easily fatigable, and apprehensive worriers. Patients with low harm avoidance are often confident, relaxed, optimistic, carefree, uninhibited, outgoing, and energetic.

Reward dependence (RD) can refer to differences in resistance to extinction of previously rewarded behavior associated with the behavioral maintenance neural system, resistance to extinction of conditioned signals of reward or relief of punishment. Patients with high reward dependence are often ambitious, sentimental, and persistent. Patients with low reward dependence are often detached, tough minded and irresolute.

A TPQ, or true-false self-report instrument, can be used to measure NS, HA and RD. The most widely used version of the TPQ (V. IV) has 98 items and 12 subscales. Further descriptions of these factors can be found in an article titled “Cloninger's Tridimensional Theory of Personality and Psychopathology Applications to Substance Use Disorders,” by Matthew Owen Howard et al., published in the Journal of Studies on Alcohol (Vol. 58, 1997), the entire disclosure of which is incorporated herein by reference. Also incorporated by reference herein in its entirety is a book titled “Personality and Psychopathology (American Psychopathological Association Series)” edited by C. Robert Cloninger.

Personality factors may be used by real-time clinical evaluation system 100 as predictors of medication selection and preference. In one embodiment, these measures may be adapted from a validated research model of Temperament initially developed by Cloninger in the late 80s and 90s.

There is a biological substrate to some aspect of personality differences. Cloninger's conceptualization and research provides a validated model for linking personality and neurochemical variables. This bridge of neurochemical correlates of personality provides an index to medication specificity. It constitutes a coherent, verifiable and testable hypothesis that specific domains of personality are relevant to medication preference—separate from the standard concepts of efficacy of treatment for attention. A medication may work to enhance attention with few overt side effects, but not be “liked” by the patient. The reason for a specific medication may seem very subtle and subjective. Patients often describe this in words not often asked by psychiatrists. Patients tend to like and continue to use a medication that works (enhances attention), so long as it also enables them to still “feel like myself”

What makes the concept of personality relevant and measurable in relation to treatment with stimulant medication, for example, is the link of these medications to specific neurotransmitters that selectively impact norepinephrine (NE) and dopamine (DA). Though less relevant to stimulants but highly related to antidepressants, there is also a bridge to serotonin (SER). It may be a short leap to also connect these neurotransmitter mechanisms to personality traits and temperament. This connection between personality and chemistry was first articulated by Cloninger in the late 80s.

Cloninger's model defines a connection between temperament and neurobiology that has been validated and refined in more detailed research and publications. For example, in Cloninger's model, norepinephrine (NE) is associated with Reward Dependence (RD). High RD is associated with being ambitious, sentimental and persistent whereas Low RD is associated with being detached, tough-minded and irresolute. The impact on task performance suggests that high RD individuals are likely to be more ambitious, vigorous and responsible in getting things accomplished. Medications that act intensively on NE, such as Vyvanse, are likely to have greater effect on these personality characteristics and enhance meaningful productivity in pursuit of success.

Elevated NE and high RD are likely associated with the patient being interpersonally loyal and committed to old friends, sustaining long-term relationships, remaining committed to concepts, ideas and attitudes that have a long personal history. Such patients are unlikely to change core ideas or beliefs and tend to do things they already know and are familiar with. That is, the patient may be inclined to take care of what they have and to preserve and reinforce what they know.

By contrast, Novelty Seeking (NS) is more reflective of Dopamine (DA). High NS is associated with impulsiveness, exploratory behavior, excitability, reactivity (quick temper), and excessive, extravagant or disorderly behavior. In contrast, Low NS individuals are more reflective and more ridged, less flexible, and more loyal. They tend to be more orderly, persistent and slow-tempered (less reactive and more emotionally stable). Accordingly, medications that act predominantly on DA, such as methylphenidates (Concerta, Ritalin and Focalin), tend to be more inhibitory, calming and stabilizing. Therefore, they are more appropriately used in individuals who need this calming effect. For example, they may help hyperactive children be calmer, quieter, and less overactive, disruptive, and more productive. When applied to Task Performance, these medications facilitate tasks that require more consistent, stabile, repetitive activity. Thus, if one has to repeat a familiar task over and over (as opposed to learning a novel task) these DA-ergic medications are more likely to be effective.

Patients who are experiencing NS tend to seek new friends and associates, are interested in novel, unfamiliar activities, are willing to explore and embrace new ideas and change and are eager to engage in new tasks. They may get bored easily with familiar activities and may have difficulty completing previous tasks before starting new ones.

Harm Avoidance (HA) and behavioral inhibition are personality traits more reflective of serotonin (SER). High HA is associated with being cautious, apprehensive, inhibited in task performance and being socially more fearful and shy. Cognitively, these individuals are likely to be more worried and obsessive.

Patients who are experiencing HA tend to be cautious, tense and apprehensive and avoid criticism or conflict. They would rather be compliant and seek to please others and avoid trouble, strive to know what is expected of them, and are attentive to rules. They wish to embrace ideas that are permanent and consistent with prior thoughts and ideas. They tend to do activities they already know and are reluctant to take on unfamiliar tasks or adventures that are novel and require learning new skills. They seek to stay safe, familiar and avoid risk.

Although not always critical to the primarily effects of psychostimulants, serotonin may be a relevant secondary modifier. There is data (Sulzer, et al.) that demonstrates that increases in NE prompt compensatory increases in SER, and vice versa. This finding led to the core hypothesis that underlies some of the impetus to development of new generations of SSRI antidepressants, as applied by Herbert Meltzer (Vanderbilt). Relevant here is that measures of serotonin could be conceptually relevant. Additional description can be found in the following, each of which is incorporated by reference herein in its entirety: (1) “Noradrenergic antidepressants: does chronic treatment increase or decrease nuclear CREB-P?,” by Manier D H, Shelton R C, Sulser F J, Neural Transm. (2002; 109(1):91-9); (2) “Lack of beta adrenoceptor desensitization in brain following the dual noradrenaline and serotonin reuptake inhibitor venlafaxine,” by Nalepa I, Manier D H, Gillespie D D, Rossby S P, Schmidt D E, Sulser F., Neuropsychopharmacol. (1998 August; 8(3):227-32); and (3) an article titled “The role of serotonin in the NMDA receptor antagonist models of psychosis and cognitive impairment” by Meltzer, Psychopharmacology (Berl), (2011 February; 213(2-3): 289-305. Epub 2011 Jan. 8).

Thus, Task×Medication×Personality factors and effects, for example, can be empirically tested and validated using the real-time clinical evaluation system. In certain embodiments, such factors comprise essential variables in determining medication selection. Differences in duration of medication within clinical subtype can also be considered. Introducing the concept of a Personality×Medication connection greatly contributes to Best Fit. Cloninger has developed a questionnaire (the Tridimensional Personality Questionnaire) that helps define these personality characteristic using 98 True/False self-scored items that generates 12 subscales of typology. In certain embodiments, the real-time clinical evaluation system 100 implements the questionnaire during the evaluation process.

In certain embodiments, there is a reciprocal interaction between baseline personality traits and medication effects. The real-time clinical evaluation system 100 recognizes that a patient's fundamental personality may impact the effects of medication and reciprocally, that medications impact attitudes and behaviors that previously were considered intrinsic and fixed.

Effectiveness can be measured in several ways. For example, the effectiveness determination of the real-time clinical evaluation system 100 may take into account the task, drug, dose and time of effectiveness as factors. In addition, the real-time clinical evaluation system 100 may take into account the task, the drug and the personality of the patient.

According to one implementation, at block 540, the real-time clinical system 100 determines whether the patient is (1) on the right medication, (2) at the right dose, (3) at the right time for (4) the task the patient is performing

In certain embodiments, in order for real-time clinical evaluation system 100 to deem medication effective to treat ADD/ADHD, there must be at least a predetermined percentage (e.g., 25%, 33% or 50%) reduction of ADD symptoms and a similar improvement in task functioning for a predetermined period (e.g., at least 4 hrs). The reduction of symptoms is primarily measured against baseline measures of inattention, and secondarily against impulsivity and hyperactivity. (Note that in adults, inattention and impulsivity are the most limiting symptoms of ADD, while in children the most limiting symptom may be hyperactivity). Meeting these criteria implies that medication is effective, but may not adequately determine whether it is optimal. Optimality requires sufficient duration of action to be effective later in the day in patients who are working or studying late. The effectiveness of the medication may be considered to be task specific if there is less than a predetermined percentage (e.g., 33%) reduction in symptoms in less than a predetermined period of time (e.g., four hours). That is, the medication may not be effective for tasks that require continuous shifting or tasks requiring great depth of attention.

According to clinical experience, Vyvanse may be preferable for most patients who have to shift from one task to another (multitasking) throughout the day. Adderall may be preferable for facilitating intensive study and learning, as is frequently required by high school or college students who are reading and learning new material. Adderall also may be beneficial for adults who are performing tasks that require continuous learning and attention to detail, as is common in such professions as law or medicine. These medications are often combined for patients (e.g., of pediatricians) who typically need to do multitasking throughout the day (shifting from one task or patient to another), but then have periods when they need to study and learn new material (e.g., students) and need a booster dose of Adderall for a 2-3 hour period. The overall efficacy of a given medication may best be determined by the weekly evaluation of its general effectiveness.

Determining the right dose depends on having sufficient medication to produce not only a definable change, but a sufficient improvement in performing the task at hand usually for the full period of time such improved performance is needed. A higher dose may increase both efficacy and somewhat enhance duration of medication. However, there are limits beyond which higher doses cease having an incremental or additive benefit. This Dose×Efficacy effect will depend on the task patients are performing and on individual metabolic characteristics, such as rate of uptake and metabolism of a specific medication.

The real-time clinical evaluation system 100 determines whether the patient has the right dose of medication based on improvement in symptoms and improvement in task performance. For example, if the medication improves patient symptoms and improvement of the task being performed by the patient is greater than a predetermined percentage (e.g., 50%) then the clinical evaluation system 100 determines that the patient is taking the right dose of medication. In the alternative, if the improvement in symptoms and task performance is less than a predetermined percentage (e.g., 50% or 75%) then the clinical evaluation system 100 determines that the patient may not be on the right dose of medication.

Initial studies of the pharmacokinetics of amphetamine in children demonstrate great variability in absorption, peak level, and metabolism of stimulant medications across individuals, which are factors that impact duration of action of the medication. The optimal dose is one that is effective for the tasks being performed and that lasts long enough for task completion with minimal or no side effects. The most common side effects from stimulants include: decrease in weight or sleep, absence of headache, stomachache, or irritability and anxiety.

Two main variables govern time of effect: Onset and Duration. The major factors affecting onset are medication specific. Generally the short-acting medications (e.g., Adderall, Focalin and Ritalin) have a more rapid onset. The longer-acting medications often achieve their duration by embedding the medication onto a matrix that sustains release—and may delay onset as well. For example, Vyvanse, which has approximately 12 hr. duration, does not attain full efficacy until 1.5 hours after administration due to metabolic processes that impact absorption, onset and duration.

Accordingly, the clinical evaluation system 100 may determine that the patient is taking medication at the right time if the onset of the medical action is soon enough to perform early tasks well and long enough for tasks that the patient performs at a later time. Alternatively, the clinical evaluation system 100 may determine that the patient is not taking medication at the right time if the onset of the medical action is too late for early tasks and the duration of the effectiveness of the medication is too short for late tasks.

In certain embodiments, at block 550, after evaluating the effectiveness of the medication, one may modify, eliminate or maintain the dosage or frequency of the medication administered to the patient.

For example, concerning medication, if, based on the patient information processed by the real-time clinical evaluation system 100, the medication is deemed to be effective and lasts more than a predetermined amount of time, the real-time clinical evaluation system 100 may recommend that the patient continue using the same medication at the same dose. If the real-time clinical evaluation system 100 determines that the medication is effective but does not last long enough, the real-time clinical evaluation system 100 may recommend that a second dose be added. Finally, if the real-time clinical evaluation system 100 determines that the medication is not effective for at least a predetermined amount of time, the real-time clinical evaluation system 100 may recommend that the patient's medication be changed.

In certain embodiments, at block 560, the information determined in at least one of block 540 and 550 is transmitted to an interested party. According to one embodiment, the results are transmitted to and/or from mobile device 110 so that they may be communicated (audibly, visually or both) to user 105.

Accordingly, medication is administered to a patient to treat a disorder. Information concerning a patient on medication is collected. In certain embodiments, because the information is collected via a mobile device 110, the information can be collected in real-time. That is, the patient can enter information as he experiences various conditions instead of having to rely on memory to inform his/her prescribing physician of the medication's benefits, limitations or side effects weeks or months later. That real-time information can be analyzed to determine the effects of the medication. Further, the real-time information can be used to analyze the patient's response to the medication. This analysis can then be used to modify and/or confirm the use of medication, and recommendations in this regard may be shared with the patient.

The above-described methods and systems provide a comprehensive, integrative database that enables a patient to track the effectiveness of his/her medication for the actual tasks they are performing at the time they are doing it. This provides a vehicle to help determine whether the patient is on the right medication (e.g., best psychostimulant) at the right dose, at the right time for the tasks actually being performed.

Similarly a physician or other medical practitioner can receive directly from the system (with patient consent) or from the patient him/herself the same results, information and/or data, thus facilitating and accelerating consideration of a change of medication, dose or timing appropriate to the patient's actual needs. This data may increase the medical practitioner's certainty of medication optimization, reduce time needed to make appropriate medical decisions and enhance patient satisfaction.

In addition, pharmaceutical companies may be able to use the independent data collected by the system to more narrowly and specifically target their patient/physician market. Such companies can use such information to help determine the ‘Best Fit’ of their medication to patients who have the diagnosis for which their medication is indicated. Paradoxically, the market share of a minority medication might increase if the pharmaceutical company has information that helps more clearly define the specific patient profile within the approved diagnosis that optimally responds to their medication.

Increasingly states, and more recently the federal government, are expanding their role and responsibility in health care. These entities are appropriately concerned with cost-effectiveness of treatment and reduction of waste. The above-described system and method offers the potential to enhance the specificity and selectivity of treatment for neurobehavioral or psychiatric disorders, such as ADD and subsequently related psychiatric problems.

The above-described data collection, analysis and patient/physician feed-back processes may be directly extended to other medical diagnoses.

The above-described system and method may similarly be used to validate new and existing medications in terms of their specific clinical advantage within the wide spectrum of psychostimulant medications.

The above-described system and method may connect neurobiological measures to treatment selection and outcome—e.g., Brain Imaging (Spect, PET, MRI), Brain Electrical Activity (Q-EEG—Quantitative EEG), and Molecular Genetics (connecting genetic variance and gene activity to specific symptoms, diagnoses, and medication treatment.)

It is often difficult to relate defined or suspected genes to specific behavioral components of a diagnosis. Rarely does a single gene account for the entire spectrum of behaviors or symptoms that comprise the expression of a diagnosis. Having an integrated, validated database system which quantifies the effect of specific medications on personality characteristics or behavioral symptoms, could empower genetic research to produce more practical interventions.

It should be appreciated that while the methods outlined in FIGS. 4A and 5A provide exemplary processes for clinical evaluation according to embodiments of the present invention, the underlying functionalities encompassed by these methods may be performed by any of the various entities shown in FIG. 1.

Having described methods and systems of real-time clinical evaluation according to various embodiments of the present invention, additional details of the various elements/entities illustrated in FIG. 1 are provided below.

### Central Computer

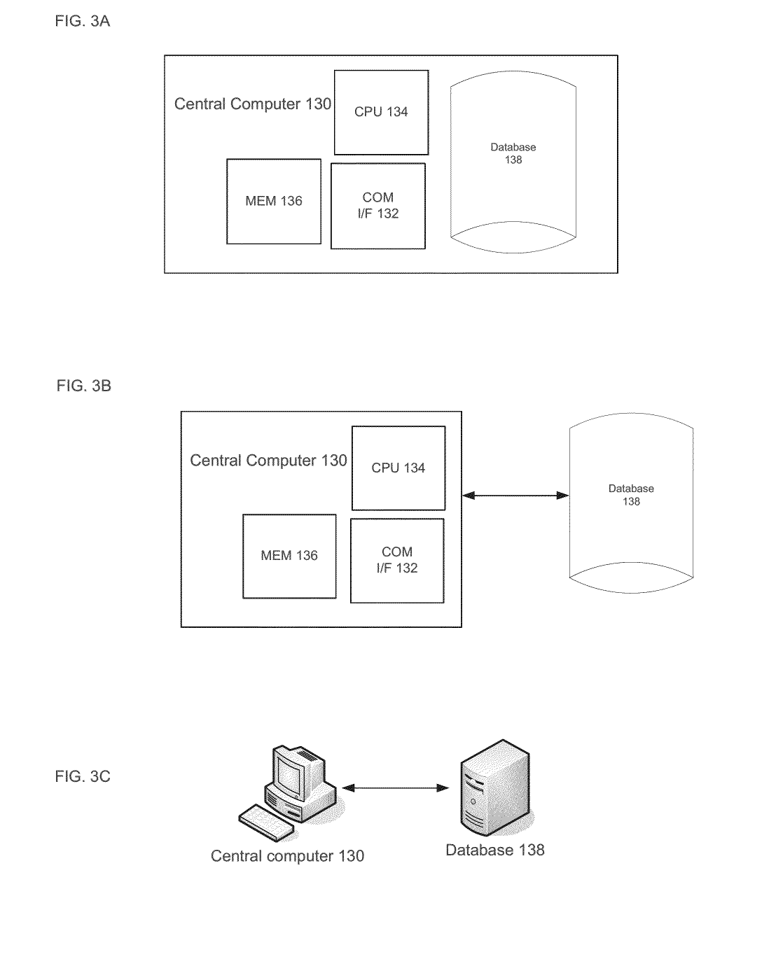

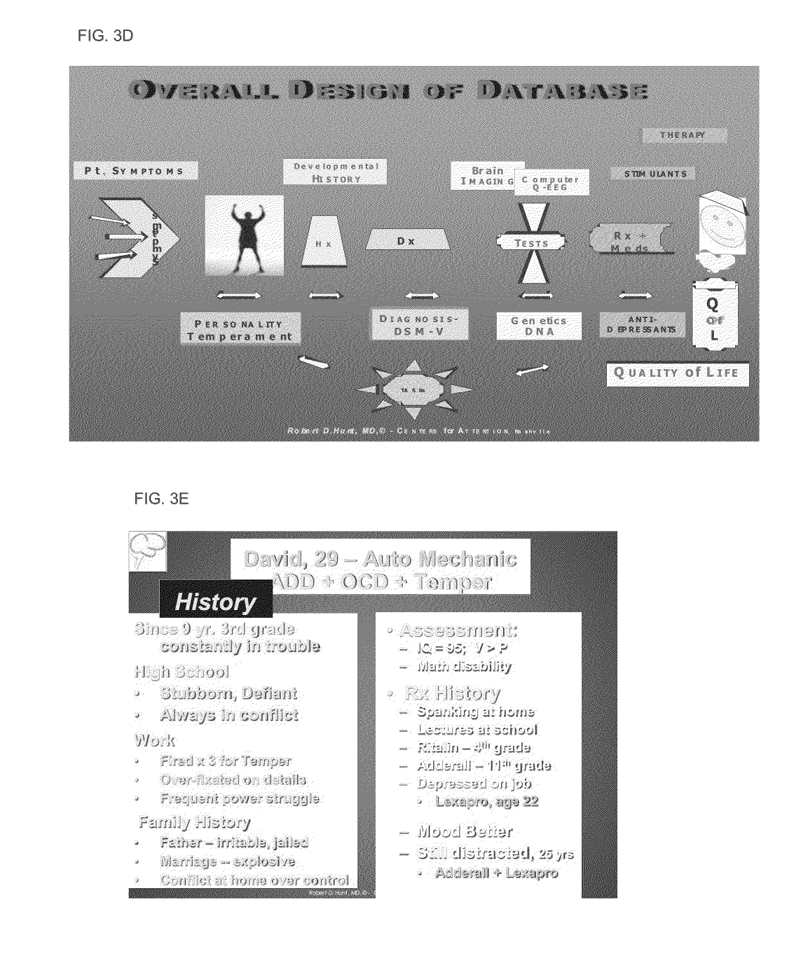

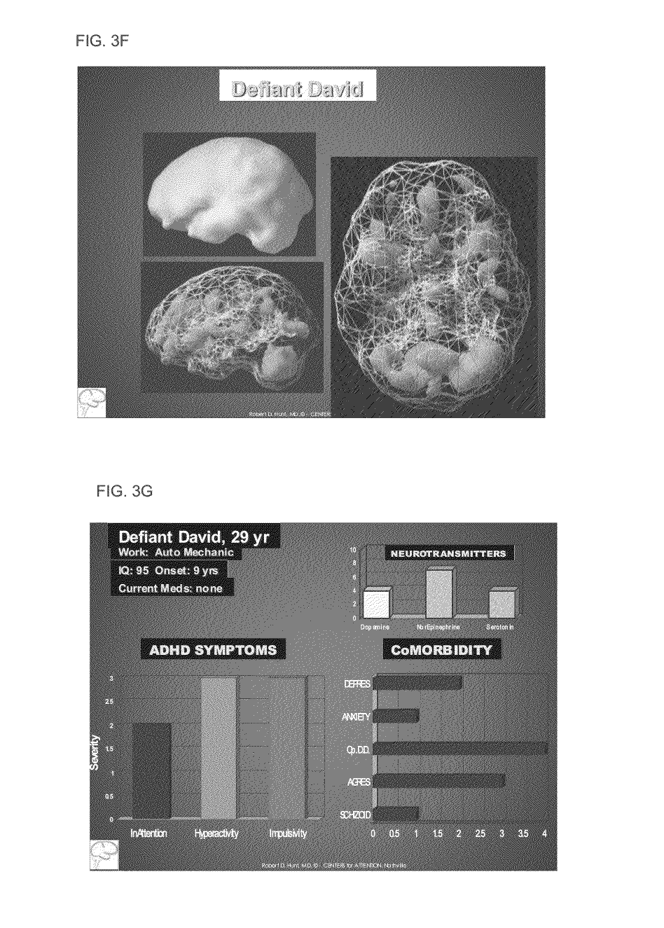

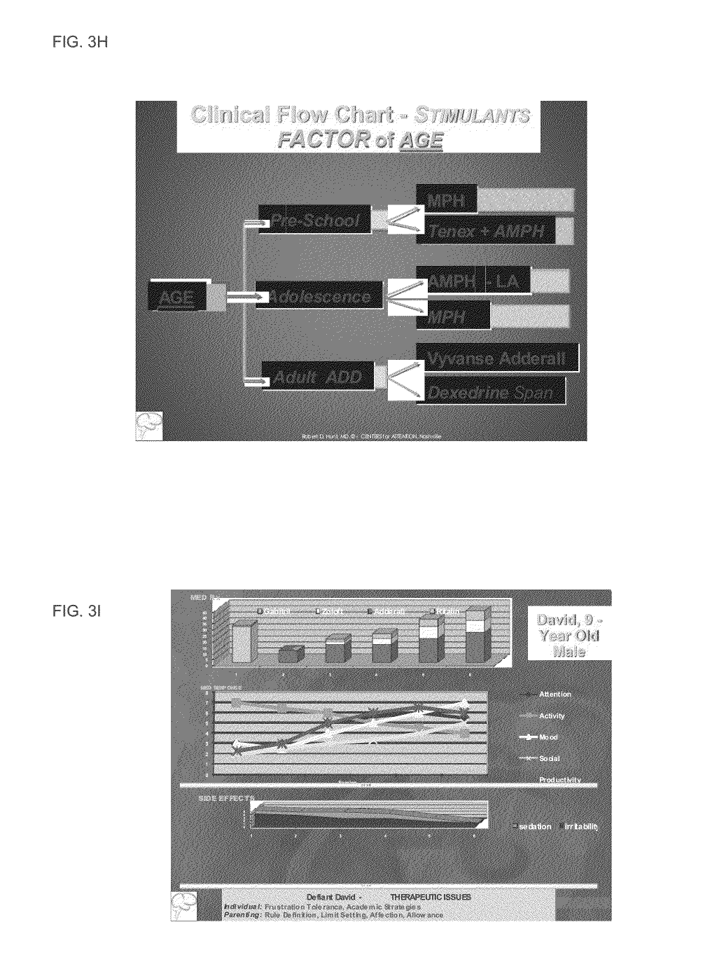

As shown in FIGS. 3A-3C, central computer 130 may be a computer, workstation or server. Central computer 130, includes input (microphone, mouse, keyboard, etc.) and output devices (speakers, display, etc.) that facilitate the input and conveyance of information. Central computer 130 runs software (custom and/or COTS) to assist in clinical evaluation. Central computer 130 may included or be associated with one or more databases 138 for storing information. For example, as shown in FIG. 3A, central computer 130 includes the database 138. In the alternative, and as shown in FIG. 3B, database 138 is an external component/device from central computer 130. For example, as shown in FIG. 3C, the database 138 may be a standalone system such as a server. The database 138 stores and organizes large amounts of patient information. The information stored in the database 138 can be stored in compliance with The Health Insurance Portability and Accountability Act (“HIPAA”) and the patient-privacy rules and regulations promulgated thereunder.

In certain embodiments, over the course of use of the clinical evaluation system 100, large amounts of patient data is generated. Database 138 in conjunction with software running on central computer 130 organizes and arranges that data to generate a comprehensive picture of a patient's clinical history. FIG. 3D illustrates a block diagram representing the type of information that may be stored in database 138 over the course of patient treatment. This information may include, but is not limited to patient symptoms, patient personality/temperament, the patient's developmental history, ability to metabolize the medication (pharmacokinetics), receptor sensitivity to given blood levels of the medication (pharmacodynamics), patient diagnosis information, the tasks performed by the patient, brain imaging, computer Q-EEG, test results, genetic/DNA information, prescription information including stimulants and anti-depressants, therapy information and information related to the patient's quality of life. In certain embodiments, the present invention simultaneously provides patient privacy and protects access to information by layering of passwords. With patient permission, selected access to an individual patient's data may be shared with that patient's prescribing physician through issuance of a patient-specific password.

Real-time clinical evaluation system 100 may use the data stored in database 138 and present that data in various ways to provide a clear picture of the patient and treatment. For example, the information stored in database 138 may be used to provide a general patient summary such as that shown in FIG. 3E. The patient summary may include information concerning the patient's school, work and family history. In addition, the summary may include assessment information, prescription history as well as information related to how the patient performed on various medications.

FIG. 3F illustrates spectral imaging that may be stored in database 138. According to one embodiment, spectral images of a patient's brain may be stored in the database 138.

FIG. 3G illustrates a graphic generated by the real-time clinical evaluation system that presents patient information related to ADHD symptoms, neurotransmitters and comorbidity. For example, the graphic shows the severity of a patient's ADHD symptoms (inattention, hyperactivity, impulsivity) as well as the relative severity of depression, anxiety, oppositional defiant disorder, aggression and social function. Further, the graphic shown in FIG. 3G indicates the amount/presence of neurotransmitters such as dopamine, norepinephrine and serotonin.

FIG. 3H illustrates a clinical flow chart for displaying the prescribed stimulants for a patient as a factor of age. For example, information stored in the patient database throughout the patient's life is used to generate a graphic of the patient's clinical history for each stage (pre-school, adolescence, adulthood) in the patient's life.

FIG. 3I illustrates several graphics that track a patient's response to medication, treatment and side effects over time. For example, information stored in database 138 can be used to chart the amount and type of medication prescribed for a patient over time. In addition, a patient's response (attention, activity, mood, social, productivity) to the prescribed medication and its side effects can be tracked over time. This allows a physician or other medical personnel with access to the database to acquire a clear snapshot of patient data.

### Mobile Device

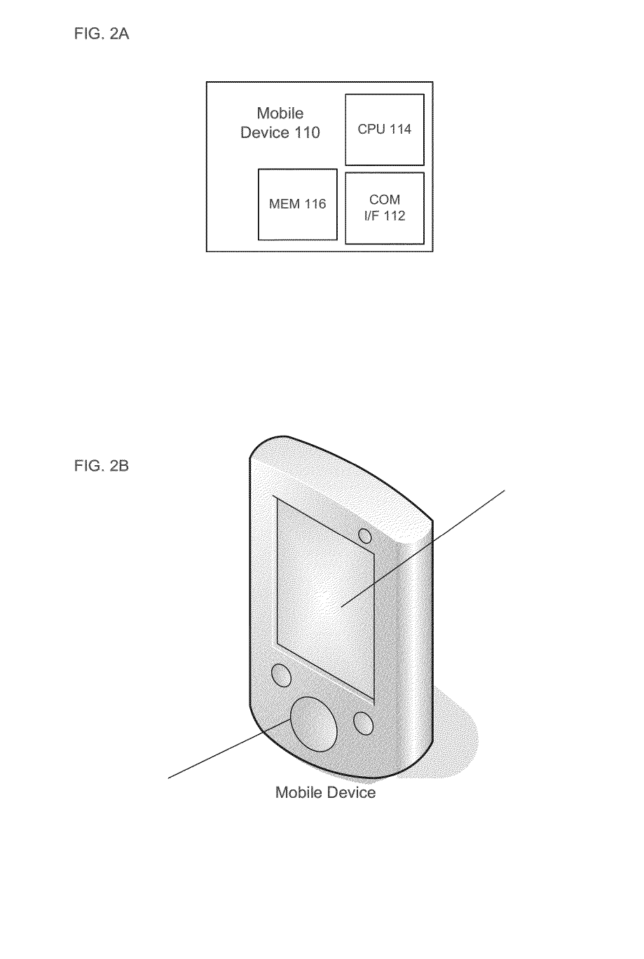

As shown in FIGS. 2A and 2B, mobile device 110 may be an electronic device capable of transmitting a signal. Mobile device 110 may include, or be associated with, a computing device, such as a portable computer, tablet device, a personal digital assistant (PDA), smart phone, cellular radio telephone, mobile computing device, touch-screen device, touchpad device. According to one embodiment, mobile device 110 is a smart phone (See FIG. 2B) running a real-time clinical evaluation application (“APP”) or software. The mobile device 110 may have a display 113 and input 115. For example, mobile device 110 may be an iPhone®, iPad®, Blackberry, Android device or other model having similar capabilities.

To provide for interaction with a user, the systems and techniques described herein can be implemented on the mobile device 110 with a display device (e.g., a LCD (liquid crystal display) or touch screen) for displaying information to the user and a keyboard and a pointing device (e.g., a mouse, stylus or a trackball) by which the user can provide input to the mobile device 110. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

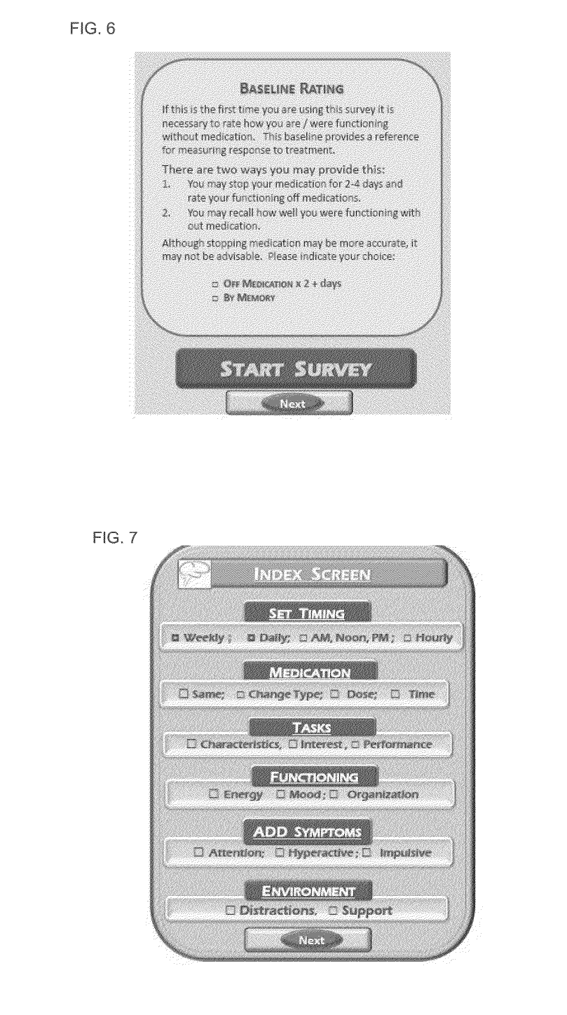

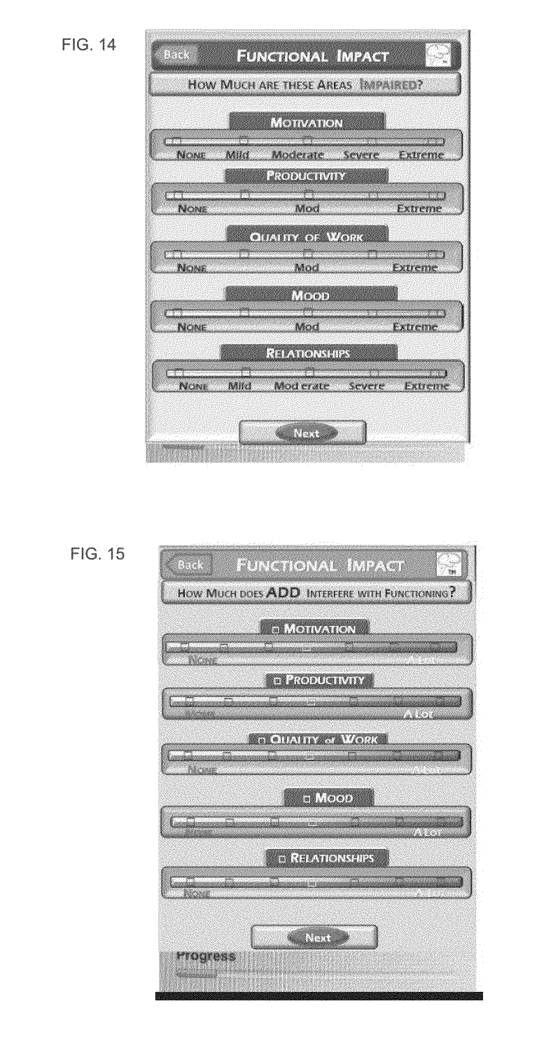

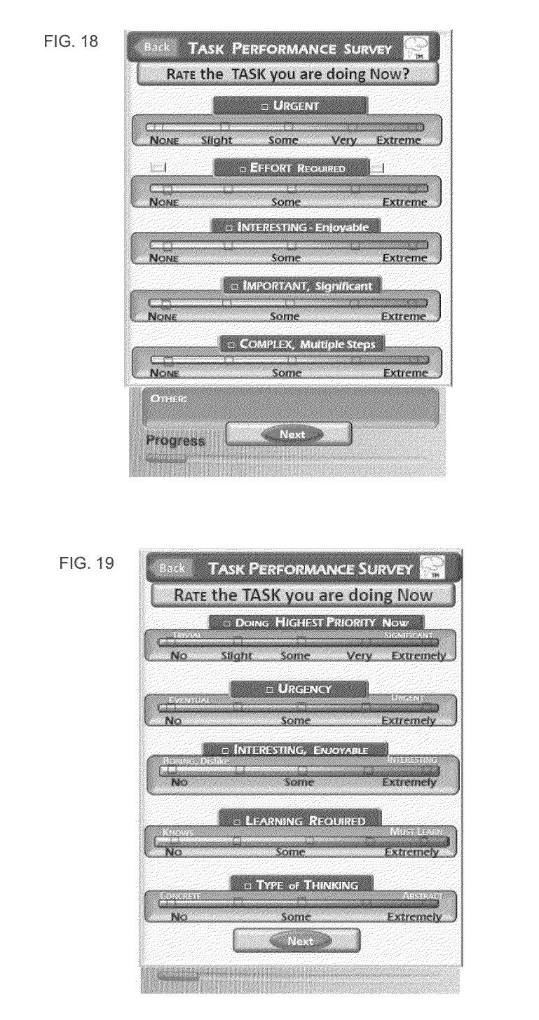

FIGS. 6-39 illustrate screenshots of a user interface presented to a user via display 113 for capturing patient information for storage in database 138 and “Best Fit” analysis. The mobile device 110 may run software or an APP for presenting a user with the illustrated screenshots, which allow the mobile device 110 to record patient information for later analysis.

FIG. 6 illustrates a screenshot of a user interface for recording information related to how a patient was performing without medication. As shown, the user is presented with the option of providing baseline information based on being off medication for a predetermined period of time or by memory of their functioning and symptoms before treatment. Prior to beginning treatment, the patient may be asked to conduct an extensive computerized patient self-rating of their symptoms and impact on functioning at work, school, and home. In the event that such pre-treatment information is not available, the system provides sufficient baseline, pretreatment data for a definitive comparison of medication response, duration, and side effects.

FIG. 7 illustrates a screenshot of a user interface of the real-time clinical evaluation system 100 for recording information related to timing, medication, tasks, functioning, ADD symptoms and environment. The timing may be set to weekly, daily, AM, Noon, PM, hourly, every 2 hours, every one half hour, etc., throughout the day. The medication setting may be set to same, change type, dose and time. The task option may be set to characteristics, interest and performance. The functioning option may be set to energy, mood or organization. The ADD symptoms option may be set to attention, hyperactive, or impulsive. The environment option may be set to distractions or support.

FIG. 8 illustrates a screenshot of a user interface of the real-time clinical evaluation system 100 for recording information related to a psychiatric diagnosis of a patient. For example, the user may select one or more of ADD/ADHD Attention defiant, ODD oppositional defiant, Mood Disorders including—depression, dysthymia bipolar disorder, anxiety disorder, ORE—Over-Reactive emotion, TD/LD—thought disorder, learning disability or OCD—obsessive compulsive disorder.

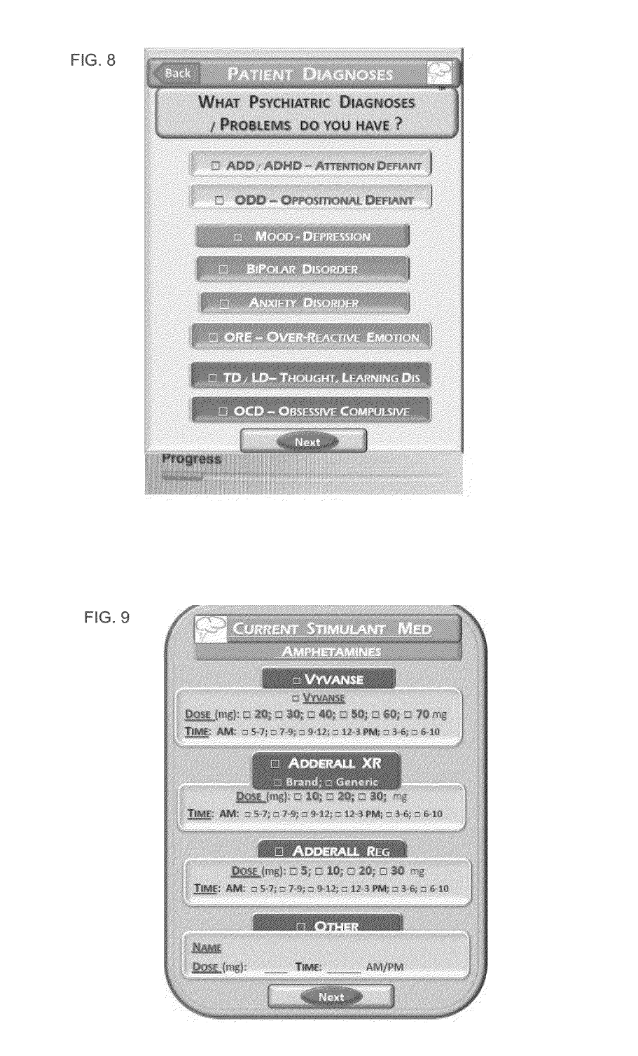

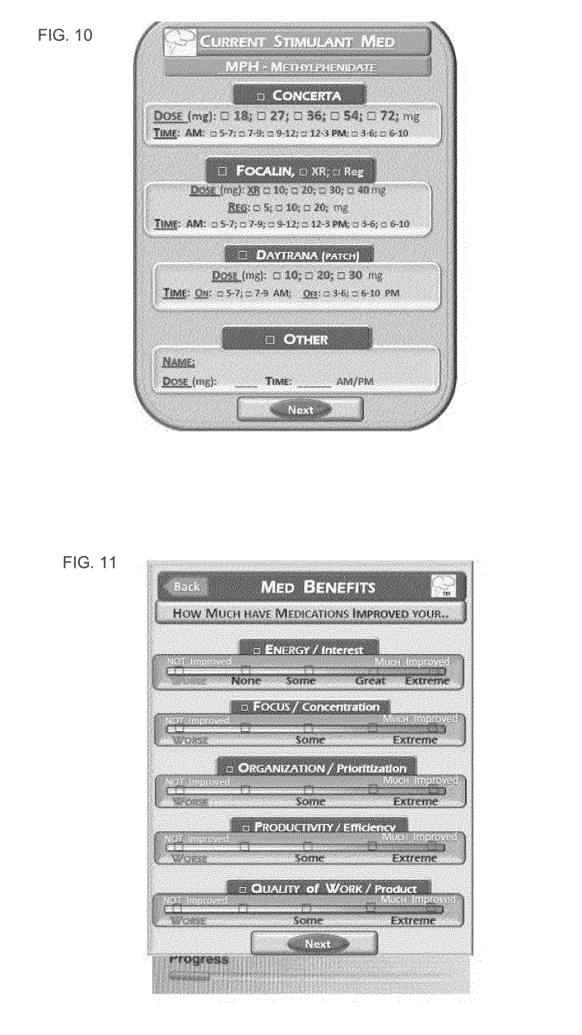

FIGS. 9 and 10 illustrate screenshots of a user interface of the real-time clinical evaluation system 100 for recording information related to the current stimulant medication the patient is taking. For example, the user may select at least one of Vyvanse, Adderall XR, Adderall Reg, Concerta, Focalin or Daytrana. In addition, the user interface allows a user to enter dose and time information for each medication.

FIG. 11 illustrates a screenshot of a user interface of the real-time clinical evaluation system 100 for recording information related to effects on the patient that may be attributed to the patient's medication. For example, a user may indicate (e.g., using a sliding scale or selecting a numerical rating) the medication improved or made worse their energy/interest, focus/concentration, organization/prioritization, productivity/efficiency and quality of work/product.

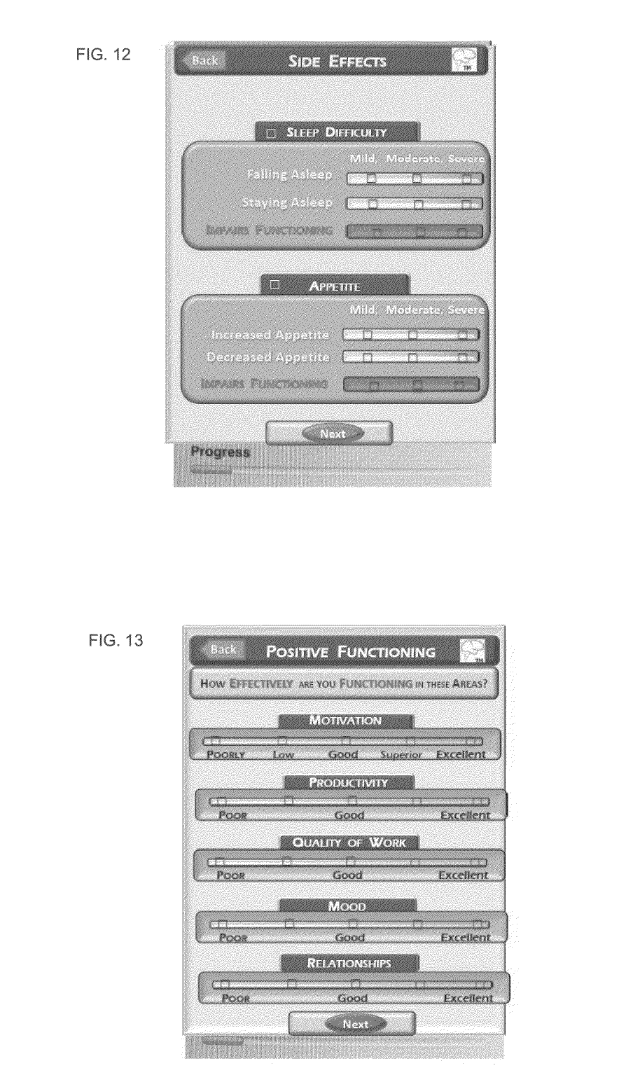

FIG. 12 illustrates a screenshot of a user interface of the real-time clinical evaluation system 100 for recording information related to the side effects experienced by a patient on medication. For example, a user may indicate (e.g., using a sliding scale or selecting a numerical rating) how the medication affects the sleep and appetite of the patient.

FIGS. 13-15 illustrate screenshots of a user interface of the real-time clinical evaluation system 100 for recording information related to how a patient is functioning. For example, a user may indicate (e.g., using a sliding scale or selecting a numerical rating) how they are functioning in the areas of motivation, productivity, quality of work, mood and relationships.

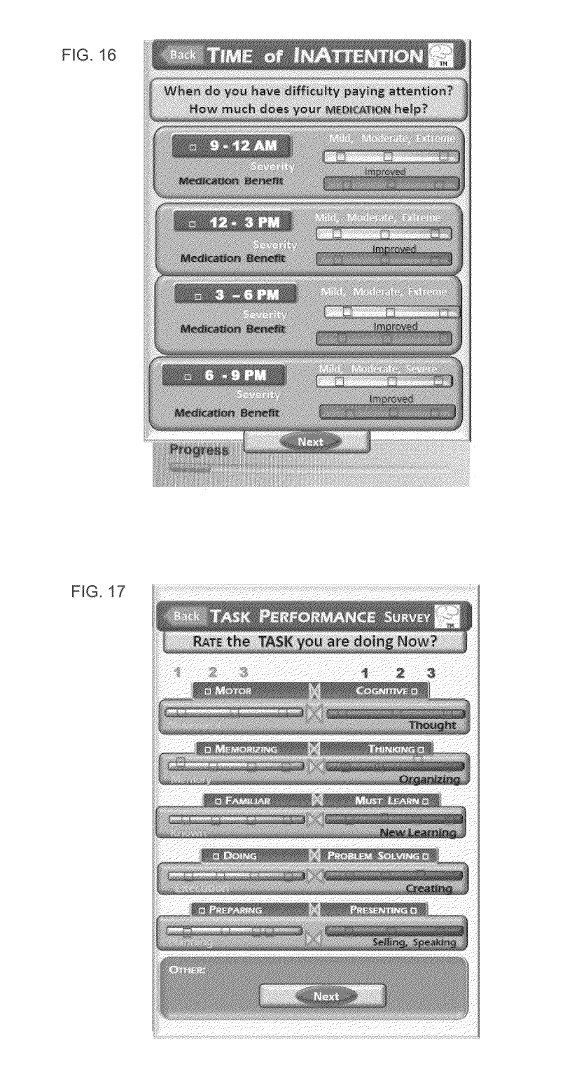

FIG. 16 illustrates a screenshot of a user interface of the real-time clinical evaluation system 100 for recording information related to the time at which a patient has difficulty paying attention. For example, for each of a predetermined span of time, the patient can indicate how much they are having difficulty paying attention and whether the medication the patient is taking improves their attention during that span of time.

FIG. 17-19 illustrate screenshots of a user interface of the real-time clinical evaluation system 100 for recording information related to a task being performed by the patient. For example, a patient may select whether the task they are performing is motor or cognitive, memorizing or thinking, familiar or unfamiliar, doing or problem solving, preparing or presenting. In addition, a patient may indicate whether the task was urgent, if effort is required, interesting/enjoyable, important/significant or complex/multiple steps. Further, the user may indicate if they are doing a high priority task and the type of learning and thinking required.

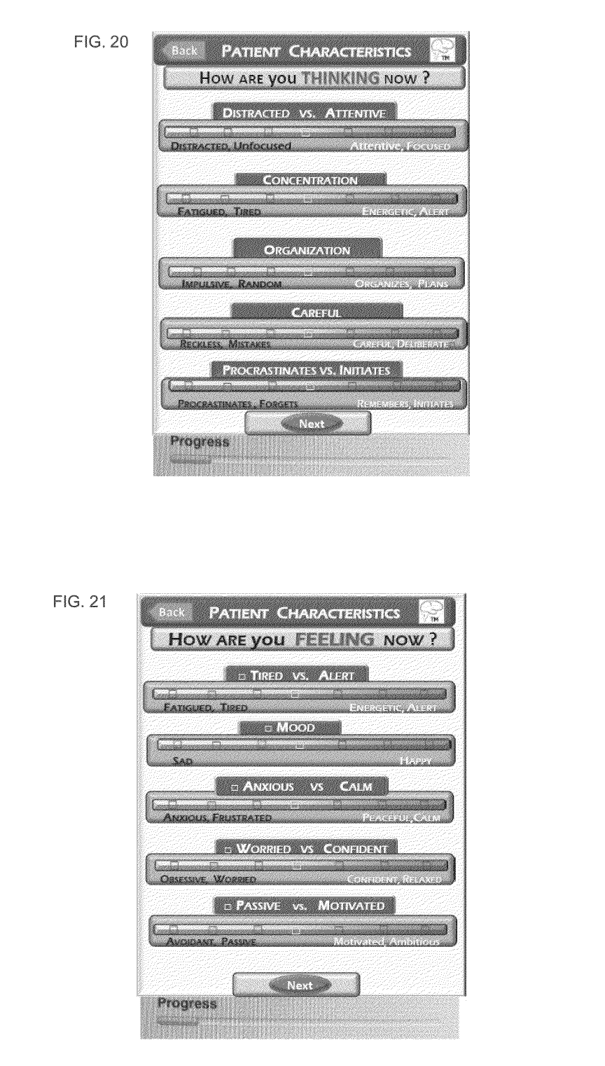

FIGS. 20-21 illustrate screenshots of a user interface of the real-time clinical evaluation system 100 for recording information related to characteristics of a patient. For example, the user can indicate whether a patient is distracted or attentive, their concentration, their organization, whether the patient is being careful and whether the patient is procrastinating or initiating. In addition, the user can indicate whether the patient is tired or alert, the mood of the patient, whether the patient is anxious or calm, worried or confident and passive or motivated.

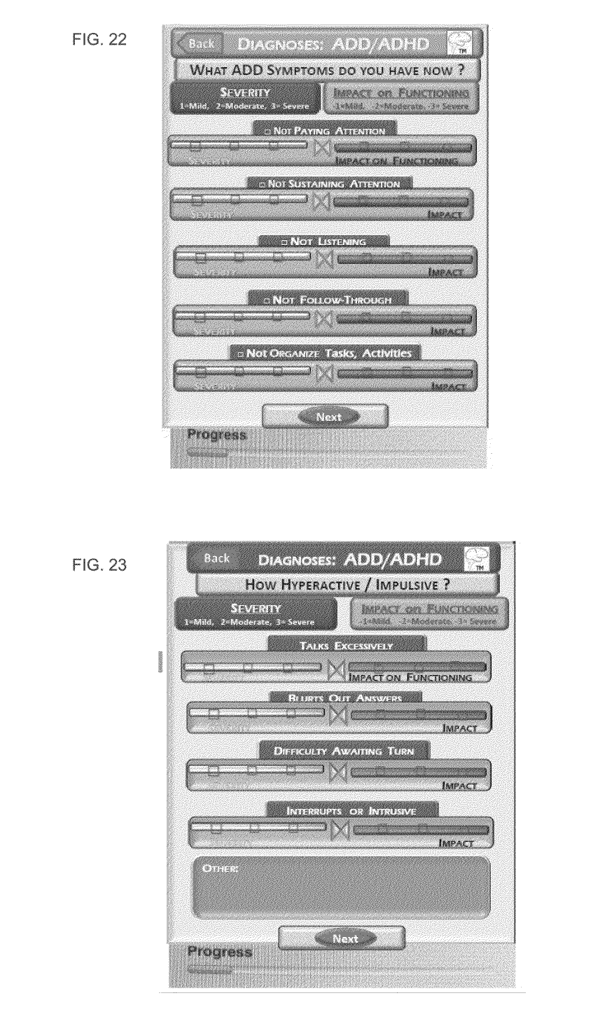

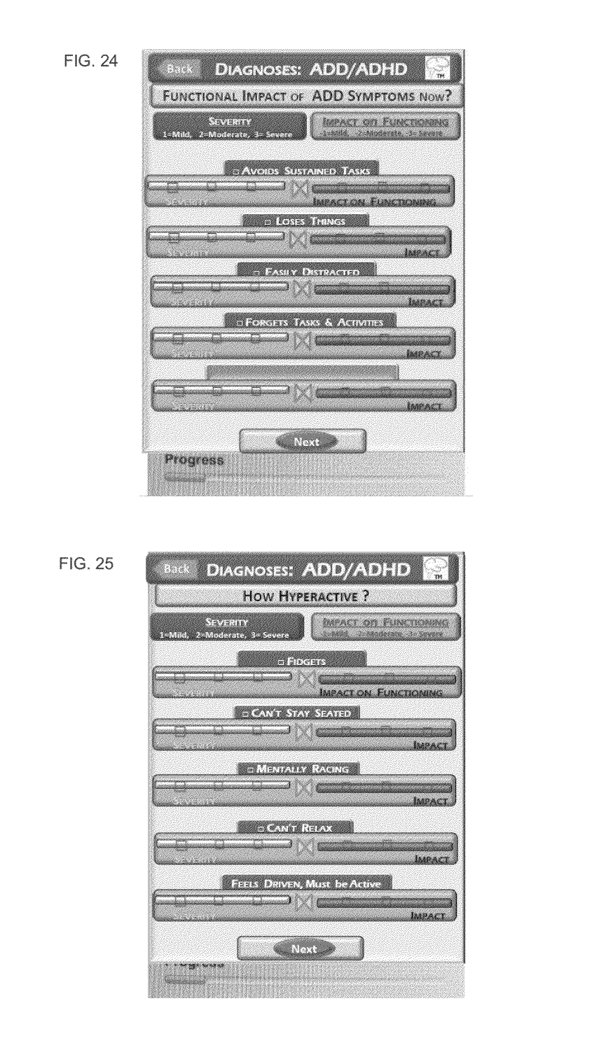

FIGS. 22-25 illustrate a screenshots of a user interface of the real-time clinical evaluation system 100 for recording information related to a diagnoses of a disorder. Each user interface allows the user to indicate the severity and impact of listed symptoms. The listed symptoms include, but are not limited to attention, listening, follow-through, organization, talking excessively, blurting out answers, having difficulty awaiting turn and whether the patient interrupts or is intrusive. In addition, the user interface shown in FIG. 24 allows a user to indicate the impact of ADD symptoms on a patient including whether the patient avoids sustained tasks, loses things, is easily distracted and forgets tasks and activities. The user interface in FIG. 25 allows a user to indicate how hyperactive a patient is including whether the patient fidgets, cannot stay seated, is mentally racing, cannot relax or feels driven (must be active).

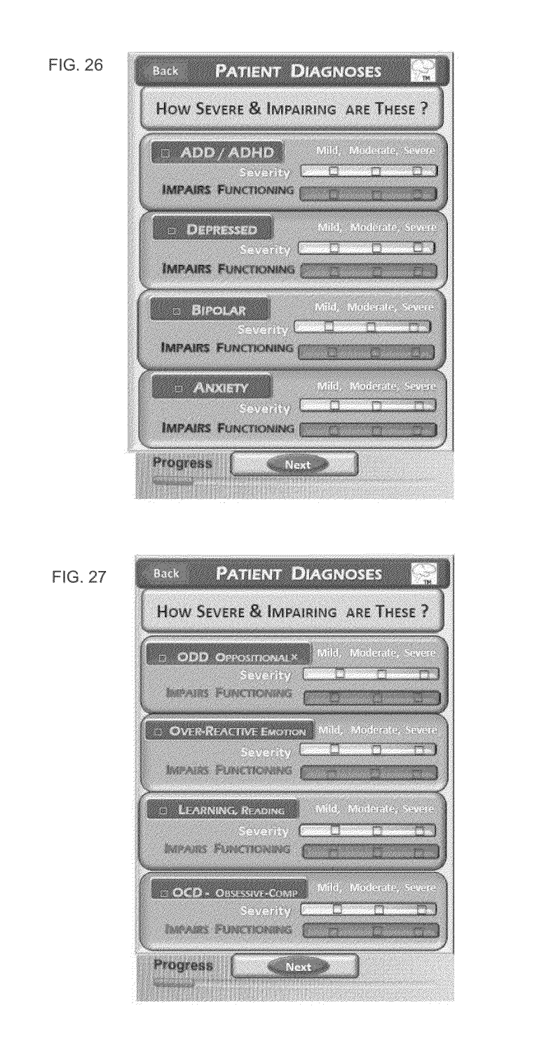

FIGS. 26-27 illustrate a screenshot of a user interface of the real-time clinical evaluation system 100 for recording information related to a patient diagnosis or impairment. For example, for each disorder (e.g., ADD.HD, Depression, Bipolar, Anxiety, ODD, over-reactive emotion, learning, reading or OCD) the user interface allows a user to indicate the severity and how much the disorder impairs a patient's functioning.

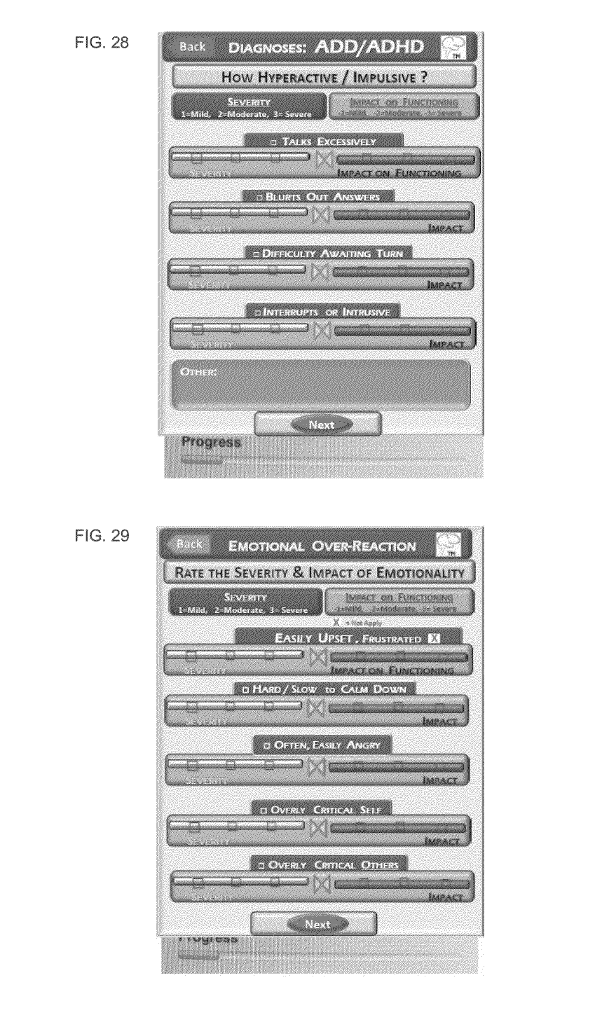

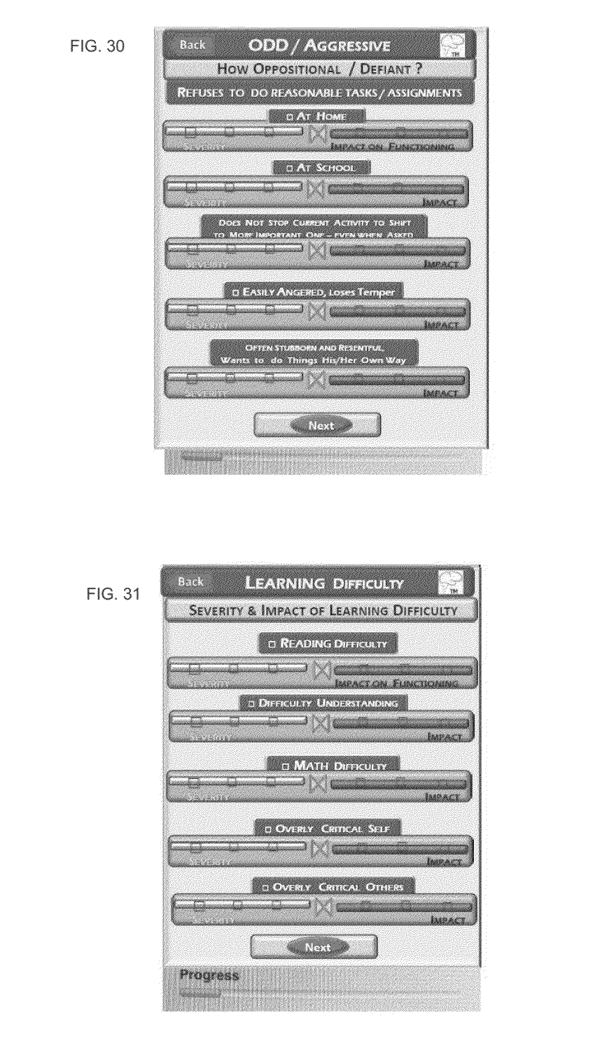

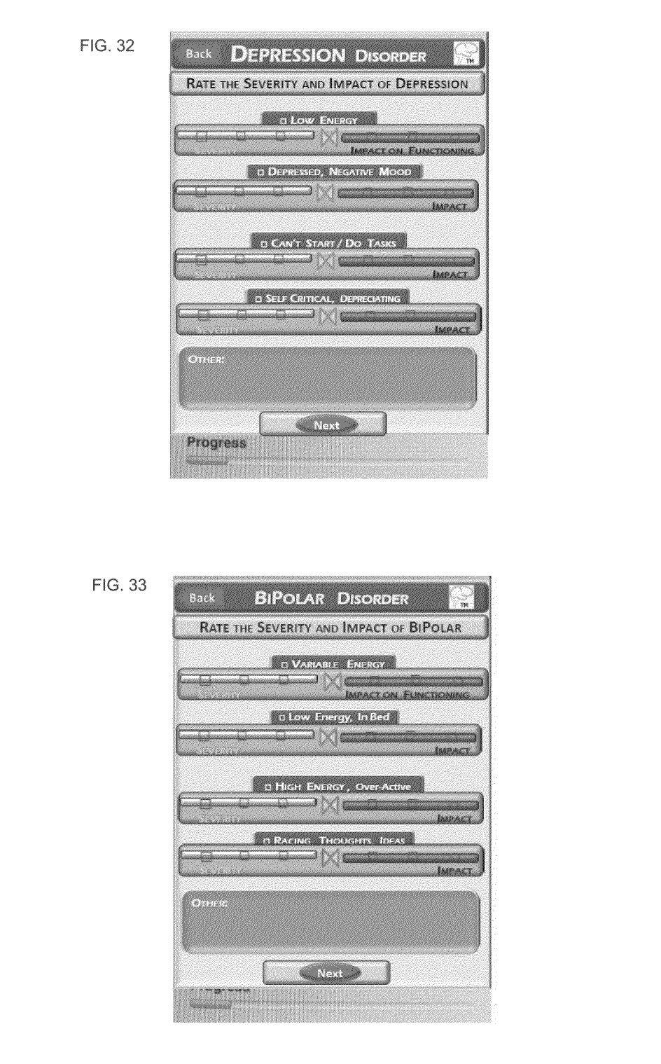

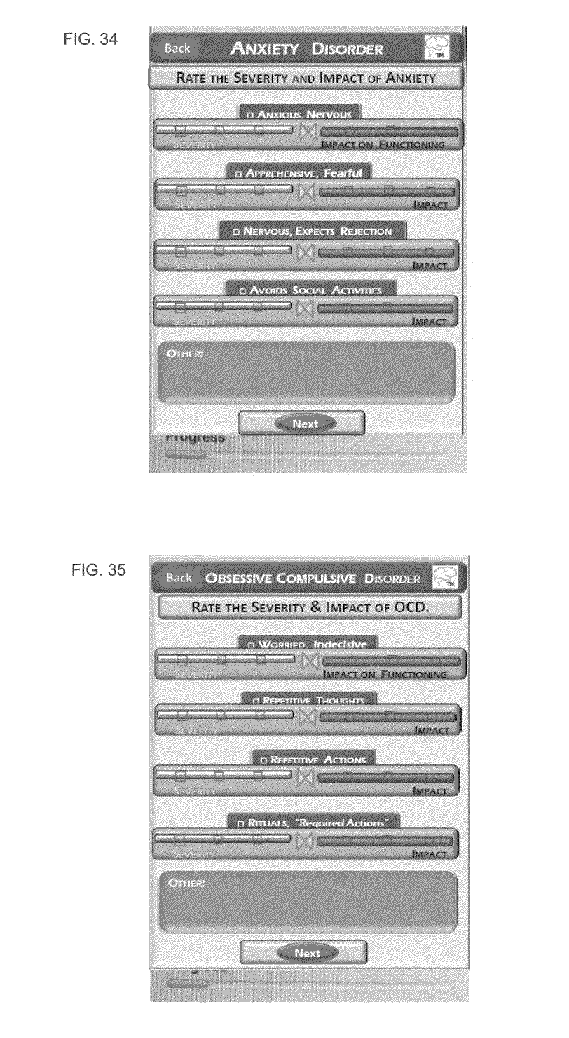

FIGS. 28-35 illustrate a screenshot of a user interface of the real-time clinical evaluation system 100 for recording information related to entering more specific characteristics related to a specific disorder. Specifically, FIG. 28 is a screenshot of a user interface for allowing a user to input information related to a patient's hyperactivity. That information may include whether the patient talks excessively, blurts out answers, has difficulty waiting their turn or interrupts or is intrusive. The screenshot is FIG. 29 illustrates a user interface that allows a user to record information related to the severity/impact of emotionality. For example a user can enter whether the patient is easily upset or frustrated, hard to calm down, often or easily angered, overly self critical and overly critical of others. FIG. 30 illustrates a user interface that allows a user to record information related to a oppositional or defiant patient. For example, the user can indicate where the patient is being defiant and how. FIG. 31 illustrates a user interface that allows a user to enter information related to a learning disability of a patient. For example, the user interface allows a user to record a patient's reading difficulty, difficulty with understanding, math difficulty, and whether the patient is overly critical of themselves and others. FIG. 32 allows a user to enter information related to depression. For example, the user interface can assist a user in recording the energy level of a patient, whether a patient is depressed, if a patient can't start tasks and whether the patient is self critical. FIG. 33 allows a user to record information related to a patient's bipolar disorder. For example, the user interface allows the user to record the energy level of the patient and whether the patient may have racing thoughts or ideas. FIG. 34 allows a user to record information related to a patient's anxiety disorder. For example, using the user interface a user can record whether a patient is anxious/nervous, apprehensive/fearful, expects rejection or avoids social activities. FIG. 35 allows a user to record information related to OCD. For example the user interface allows a user to record whether a patient is worried/indecisive, has repetitive thoughts, takes repetitive actions and performs rituals.

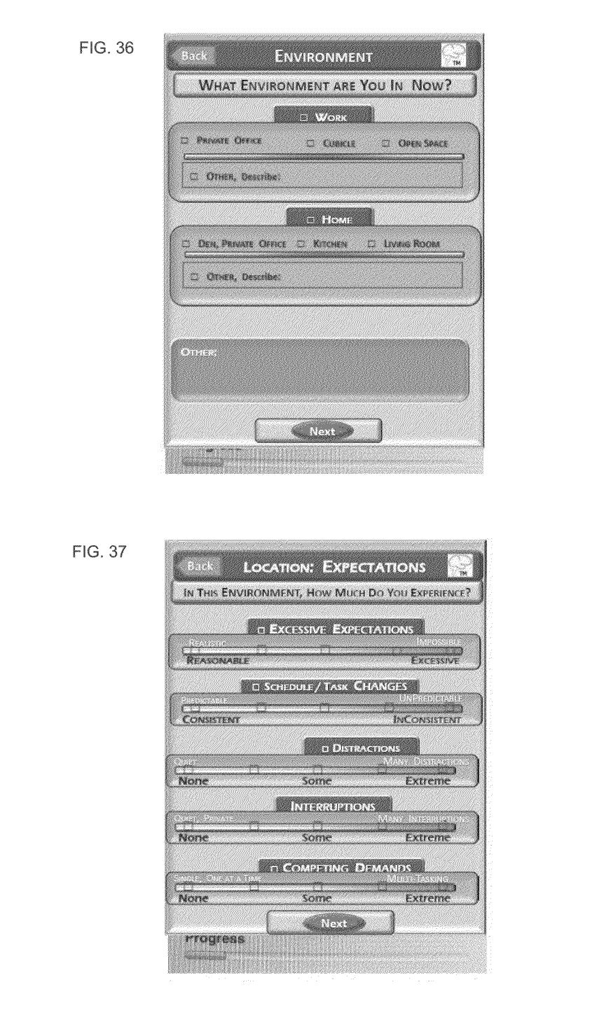

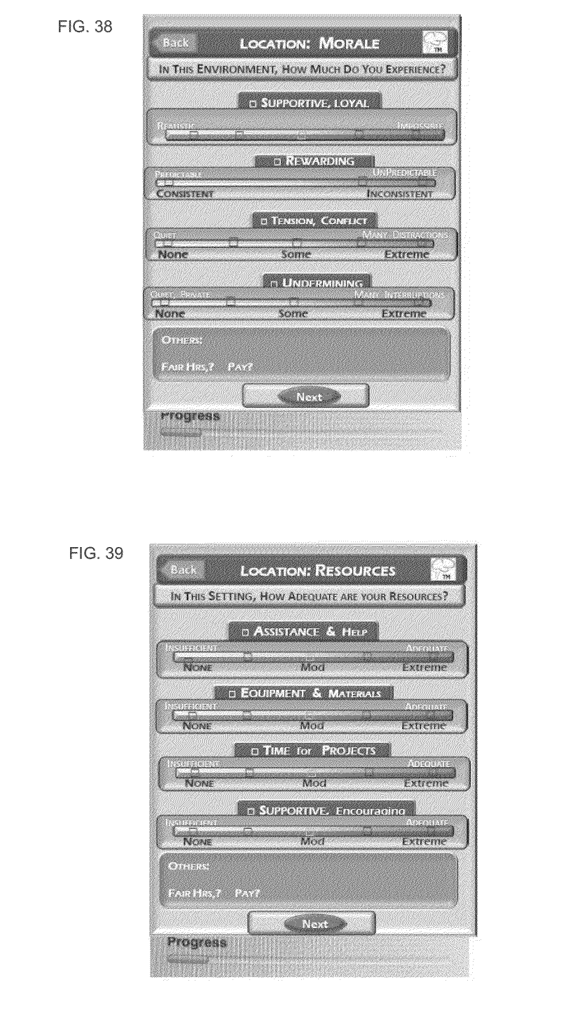

FIGS. 36-39 illustrate screenshots of a user interface of the real-time clinical evaluation system 100 for recording information related to environment. For example, FIG. 36 allows a user to record whether the environment is work or home and further specify specific rooms or settings within those two environments. FIG. 37 allows a user to specify the expectations placed on a patient in a certain environment. For example, the user interface allows a user to specify whether a patient experiences excessive expectations, schedule/task changes, distractions, interruptions, or competing demands. FIG. 38 illustrates a user interface that allows a user to specify the morale of a particular environment. Specifically the user interface in FIG. 38 allows a user to enter whether an environment is supportive/loyal, rewarding, tense/conflicted or undermining. FIG. 39 allows a user to enter information related to resources available in a particular environment. For example, a user may enter whether assistance and help are available to a patient, whether equipment and materials are available to a patient, whether the patient has time to work on projects in the environment and whether the environment is supportive and encouraging to a patient.

In some embodiments, screenshots may be provided which are configured to provide a physician with patient lists for particular selected medications. For example, one list may be provided for patients who are not taking their medication, another for patients in whom the medication appears to be working well, another for patients in whom the current medication is effective for part of the day but the current dosing is not lasting as long as needed, another list for patients in whom the medication is not highly effective, etc. The lists may assist the physician in making decisions about the appropriate response or adjustment for the individual patients.

In some embodiments, a user interface may be organized to separate information about symptoms and diagnoses from information about functioning. The patient may be prompted to describe improvement in symptoms separate from describing improvement in functioning. Functioning may be separated by types of task and/or times of day. User interface elements regarding types of tasks may be configured to solicit information relating to difficulty with multitasking, difficulty with depth of attention and studying or new learning, difficulty with motivation and initiation, and/or other types of tasks.

The system described herein can be implemented using a remote website that communicates with a mobile device application. The website can be accessed by users through a web browser, web portal, the mobile device application, etc. In an illustrative embodiment, the mobile device application described herein can be a standalone application that is stored on the mobile device. Users may access the website from a mobile device, and the mobile device can exchange information with the website using any communications network known to those of skill in the art. The system can be implemented using cloud computing in which the remote website receives information, processes the information, generates results, etc. Any of the operations described herein can be performed by the website. For example, the programming and algorithms of the web site can govern the definition of patient data available to be collected, transmittal by the patient of confidential data, the receipt by the web site of the data, data storage, data sorting, data analysis, testing against norms, evaluating the degree of variance of performance from norms, identifying the source of such variance, analysis of likely impact upon patient performance of other medications, analysis of likely impact upon learning and retention effectiveness, etc. The programming and algorithms of the website can also allow the system to determine combinations of medications, recommend phasing throughout the day of the medication and dosage based on the demands of the tasks facing the patient, reporting of concrete changes in medication, dose, or time of delivery, etc. The programming and algorithms of the website can further allow the system to learn to favor medication results which have generated improved performance. As such, the system can be self-learning based upon the reinforcement model built into it. In addition, the results can be analyzed, graphed, and made available to the patient and his/her physician through the website, through the mobile device application, via e-mail, etc.

A manual and guideline for use of the applications and web site described herein is also developed to provide guidelines for administration and interpretation of the application. Consenting patients may be asked to demonstrate the process of using the system and discuss the practical benefit to them of utilizing this instrument to refine their selection and dosing of medication. Many patients will utilize this process to selectively define their dose, or need for a subsequent afternoon/evening dose based on the actual tasks required that day. This allows further personalization of medication use and minimization of total dose and side effects on days requiring less attentionally-demanding tasks.

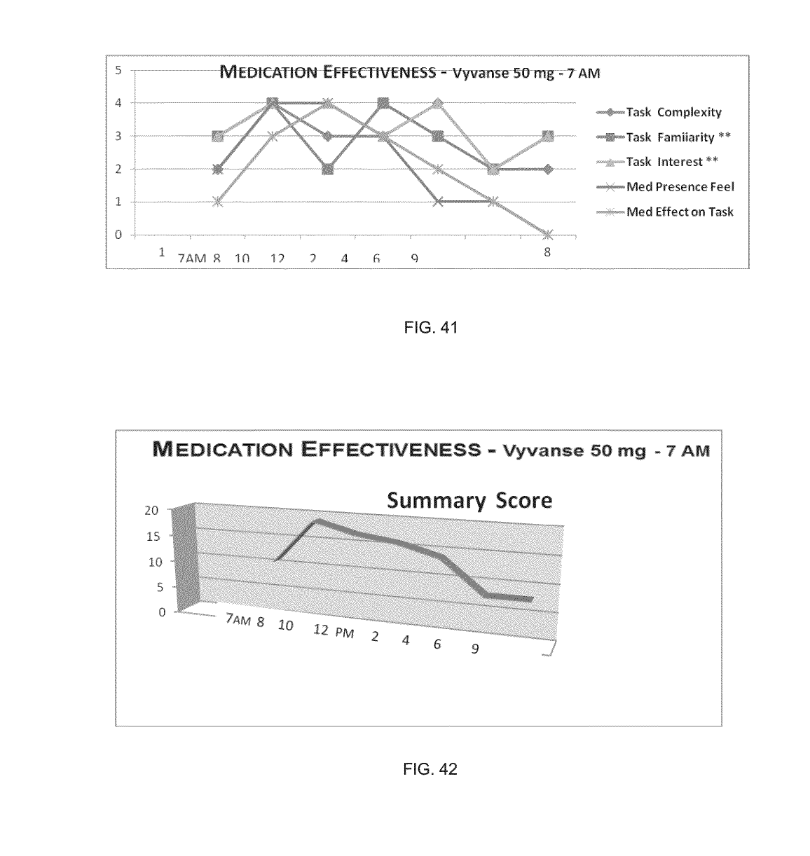

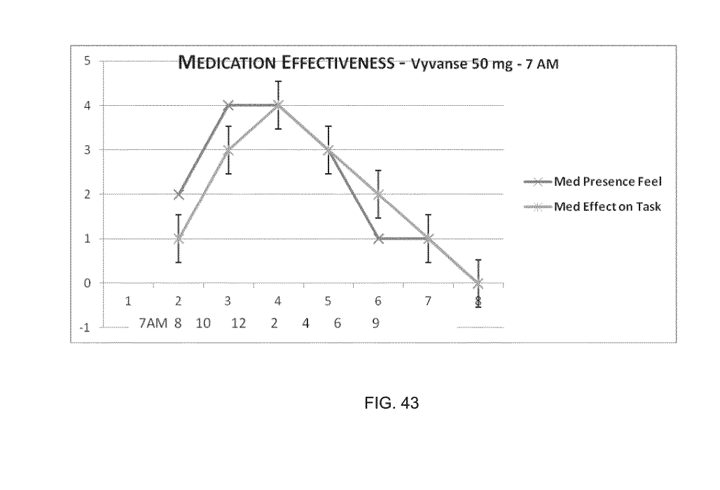

FIGS. 41-43 illustrate diagrams showing the effectiveness of 50 mg of the medication Vyvanse over a period of time based on information collected using a clinical evaluation system according to an exemplary embodiment. FIG. 41 illustrates the effectiveness of the Vyvanse over time with respect to five separate factors: task complexity, task familiarity, task interest, whether the patient feels the presence of the medication, and the effect the medication has on the performance of the tasks. FIG. 42 provides a summary score over the period of time based on the various observed factors. FIG. 43 illustrates the same data curves as shown in FIG. 41 regarding whether the patient feels the presence of the medication and the effect the medication has on the performance of the tasks, and those two particular curves are separated from the other information provide in FIG. 41 for ease of reference.

### Example “APP”s and Additional Example Aspects

The current invention, for example in the form of a relevant computer application or “APP” (such as used on an iPhone® or iPad®, for instance), may operate in different time frames. In one embodiment, the present invention contemplates a “more comprehensive” APP that can be used to provide baseline information. Use of this more comprehensive APP is repeated less often than daily, for example, on a weekly or monthly basis. This APP poses a spectrum of questions requiring, for example, about 10-30 minutes, such as 15 minutes, to complete. In one embodiment, this APP assesses, for example, attention and hyperactive/impulsivity sensitivity to selective medication response, differences in mechanism and duration of action among stimulant medications, as well as the relevance of these factors to personalized treatment and information, such as genetic profiles, brain scans, medical history, etc.

Such an APP can also encompass multiple concurrent diagnoses, personality factors and neurobiological measures. In certain embodiments, it samples for indices or characteristics of the environment in which the patient is performing these tasks, including distractions and reinforcements or rewards. It invites patients to score the complexity of the tasks they are performing and their familiarity, interest and need for learning of the tasks. It scores for other factors that may be affecting their attention—mood, alertness and task performance—beyond medication.

In addition to the more comprehensive APP, the present invention also contemplates a “brief” APP used more frequently by a user, such as once, several or many times a day. This APP operates in a different time frame that provides, for example, a brief presentation “hourly” (or several times a day) of fewer items (e.g., about 10 items) that quickly allows scoring of attention, impulsivity, and interest in the tasks being performed. In one embodiment, this APP prompts for scoring at least 4× or 5×/day, for example, once at about 8 AM (or ˜1 hr. after taking medication), then again at about 11 AM (about 3-4 hrs. post dose) and then again at 2 PM and 6 PM, as an index of duration. A final rating is performed about 9 pm if the patient is doing tasks (such as homework or office work) that require attention late in the day.

One rationale for the brief APP, either alone or in combination with the more comprehensive APP, is that it provides feedback in real time regarding the action and duration of the medication taken by a patient, It also offers suggestions for physician consideration regarding whether, for example, a second additional dose of a short or acting medication is needed late in the day or the option of an alternative longer-acting medication in the morning. This system also provides a vehicle of tracking whether “habituation” or reduced responsivity emerges with regard to a given medication and dose over time. Clinically, patients often require a slight increase in dose or a second dose in order to accommodate for changes in their metabolism or their brain's response to these medication. Such accommodation may reflect emergent changes in pharmacokinetics (absorption, metabolism and excretion) or in pharmacodynamics (the response to a specific dose or blood level of medication).

Established Scales.

The present invention, such as in an APP format, may be validated, confirmed, and/or include other aspects that supplement relevant information obtained. For instance, one may use the present invention in conjunction with core standardized, highly recognized and validated rating scales, such as ADDRS and the Vanderbilt Scale. ADDRS is an 18 item scale widely used in adult ADD diagnosis and treatment. This scale uses the 18 items and approximate vocabulary of DSM-IV diagnostic criteria for adult ADD, and rates the items on a 4 point sale, where 0=none; 3=often/severe. The Vanderbilt Scale assesses about 35 items and includes many concepts from DSM-IV and integrates items from the Conners' Scale.

In one embodiment, integration of the present invention and known established rating scales is accomplished by asking a group of patients (e.g., ˜50 patients) to complete a weekly (and/or more or less frequently used) APP of the present invention in concert with assessing ADDRS and Vanderbilt scales and two other scales on 4 consecutive weeks during random assignment of 2 weeks off and 2 weeks on active psychostimulant medication vs. placebo or no medication. The present invention contemplates a correlation of >0.7 between ratings on the APP of the present invention and these standardized measures. Subscale items will also be analyzed for inattentive as well as hyperactive-impulsive items.

Objective Observer Ratings.

In one embodiment, the invention asks a spouse or (for children) parents to perform a weekly rating of the patient to correlate with the patients self-rating. Research suggests that patients tend to rate their level of impairment as less severe than other external observers.

Video Recording.

Video cameras and computers, such as the iPhone®, iPad® or comparable “smart tablets,” which have build-in cameras, can also be used to validate or supplement information generated from an APP of the present invention. For instance, one exemplary system asks patients to generate a series of 15-20 minute video recording of themselves in 15-20 minute segments at least 3-5×/day and simultaneously rate their own behavior. Trained observers score the videos, for example, in terms of degree of apparent attention to task based on body language and movement, head movement and eye tracking. The system can assess continuity of task performance without interruptions, accounting for the extent of environmental distractions. In addition, the system can track task complexity, interest and attentional demand. The system can compare self ratings with objective observer's ratings.

Similarly, a recent technology called “Kinetic” applies gaming technology to video and can score body moments. In one embodiment, the present invention uses such technology to assess an index of hyperactivity, inattention and depression, and allows further evaluation of medication response. Likewise, virtual reality systems are current used, such as in military operations and flight simulation. In the present invention, such systems may be used as part of the present invention to track medication response, measurement of attention, and ADD medication effectiveness.

Other Processes/Systems.

In one embodiment, a process of the present invention includes an adaptation of college entrance exam questions as relating to a measurement of attentional medication effects. The type of questions used in ACT and SAT exams include domains of math logic, reading comprehension and recall, problem and puzzle solving, Miller analogies, etc. These well-standardized and validated measures can be used as an index of attention and a measure of medication response.

Example APPs Relating to Childhood ADD.

In one embodiment, an APP of the present invention assesses childhood ADD (below age 15) and includes brief ratings by parents, such as in the AM (before school), PM (after school) and evening (about 8 PM), and consists of, for example, about 5-8 questions relating to issues of attention, organization, mood, attitude, autonomy, helpfulness—and about 5 questions on “the burden of illness”—the amount of time and effort parents must spend on organizational and directive action to facilitate their child's performance of chores, tasks, and school assignments. In certain embodiments, the APP assesses the parents' degree of trust in their child's level of autonomy and judgment in selection of friends, management outside the home, responsibility in intimidate and potentially sexual situations, extent and ease of communication in these matters. The APP can also assess, or be compared to and validated by, other relevant scales (Achenbach, Child Behavior Checklist).

An APP of the present invention can also optionally include a teacher's rating. For example, an APP can comprise a brief, e.g., 10 item, scale that assess a child's preparedness for class, attention and on-task behavior in class, degree of relevant participation in class discussions, social appropriateness, and apparent mood (looks happy, engaged, vs. depressed, withdrawn), peer likability or popularity, learning, memory, comprehension and interest and quality of participation in academics, athletics, and artistic or creative activities. In some embodiments, the teacher may be allowed to choose particular items to rate multiple times per day, and the items may be chosen specifically for the child being studied. For example, the teacher may evaluated three times per day whether the child is on task and what the child's mood or behavior is like.

Other Example APPs.

The present invention also includes APPs that adapt rating scales to measures of mood (e.g., depression, bipolar and mood instability, emotional reactivity and over-reactivity and anxiety), degree of obsessiveness and perseveration, and/or PTSD—response to trauma and neglect. The concept and impact of neglect has been under-appreciated in psychiatric assessment but is profoundly evident in many patients. An APP may track the impact of injury or trauma on physical as well as emotional or cognitive functioning. In another embodiment, an APP of the present invention uses a rating scale for aggression. In another embodiment, the present invention includes an APP for Asperger's, which addresses social remoteness, apparent diminished interactivity, poor eye contact, and excessively circumscribed interests—especially if limited to technology.

### Research Application

Various exemplary embodiments disclosed herein may be utilized to research the effects of certain treatments across multiple patients. Information collected through the APP may be anonymously aggregated and analyzed to provide patterns that may be helpful in treatment selection for other similar patients. By utilizing anonymous results for many individual users, the APP may enable large-scale determination of medication effects across many patients and may be used to identify patterns of effects across multiple patients based on characteristics relating to the patients and/or medication. Application of the aggregation and analysis method may lead to better medication selection within a particular diagnostic category, as may be evidenced by improved efficacy and/or compliance with treatment. Cumulatively, a broad application of the aggregation and analysis method may enhance quality and reduce cost of psychotropic medication treatment by facilitating the selection and dosing of the optimal medication for each patient for efficiently. In some embodiments, a regression analysis may be used to differentiate predictors of optimal response (e.g., effectiveness×duration of benefit×appropriateness for task). Results from multiple patients with the same diagnosis may be analyzed to determine the continuity and variability of patients with the same diagnosis on the same medication performing relatively similar types of tasks; this information may be used to identify task-specific characteristics of specific psychostimulants. Example questions, some specific to ADD, that may be addressed using such an aggregation and analysis method include, but are not limited to:
In general, how long do each of these medications last? To what extent is this duration a function of dose, of the specific type of tasks performed, of the patients' age or weight (mg/kg), or of their duration of treatment on psychostimulants or on this specific current mediation?
In actual use over sustained treatment beyond 6 months, is there a change in medication efficacy or duration of action? How often does the dose need to be increased to maintain optimal effect?
Is there a difference or preference for Vyvanse when patient are performing familiar tasks that require multitasking?
Similarly, is there a preference for Adderall during tasks that demand new learning and more in-depth, sustained attention?
Does Intuniv buffer excessive emotionality and enhance frustration tolerance—thereby extending attention and task performance.
Does Strattera improve comprehension and organization of information of task performance in ADD patients who are also be taking a psychostimulant?
Are there personality characteristics that modify response to and preference for psychotropic medications?

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone, hand-held pad or tablet, or any other suitable mobile, portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of input and output. Examples of input devices that can be used for a user interface include keyboards, and pointing devices, such as mice, touch pads, and digitizing tablets. As another example, a computer may receive input information through speech recognition such as Dragon-Naturally Speaking or in other audible format.

Such computers may be interconnected by one or more networks in any suitable form, including a local area network or a wide area network, such as an enterprise network, and intelligent network (IN), or the Internet. Such networks may be based on any suitable technology and may operate according to any suitable protocol and may include wireless networks, wired networks or fiber optic networks.

Any computer discussed herein may comprise a memory, one or more processing units (also referred to herein simply as “processors”), one or more communication interfaces, one or more display units, and one or more user input devices. The memory may comprise any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to and/or receive communications from other devices. The display unit(s) may be provided, for example, to allow a user to view various information in connection with execution of the instructions. The user input device(s) may be provided, for example, to allow the user to make manual adjustments, make selections, enter data or various other information, and/or interact in any of a variety of manners with the processor during execution of the instructions.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. The terms “software” and “non-transitory computer-readable medium” also encompass an application (or “APP”) for a computer, such as a mobile device, e.g., an iPhone®, or iPad® or similar type of device. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for real-time clinical evaluation of effects of a medication for the treatment of a disorder in a patient, comprising:
receiving information concerning: (i) a medication being taken by a patient for the treatment of a disorder; (ii) at least one effect of the medication on the patient; (iii) characteristics of a current task being performed by the patient; and (iv) a current baseline of the patient; and
calculating or providing, by one or more computers, results indicative of an evaluation of effects experienced by the patient upon taking the medication based on the received information.

2. The method of claim 1, further comprising:
obtaining the received information at a central computer;
storing the received information in a database; and
analyzing the patient's response to the medication based on the received information and patient information previously stored in the database.

3. The method of claim 2, wherein the patient information previously stored in the database comprises information relating to at least one selected from the group consisting of the patient's medical history, prior symptoms, developmental history, ability to metabolize the medication (pharmacokinetics), receptor sensitivity to given blood levels of the medication (pharmacodynamics), personality, temperament, results from brain imaging, computer Q-EEG information, results of medical, diagnostic or genetic testing, quality of life, use of prescription, over-the-counter or illegal drugs, and hormone levels.

4. The method of claim 1, further comprising periodically receiving the received information after the patient has been prompted by a mobile computer device to enter and transmit the received information.

5. The method of claim 1, further comprising transmitting the results to the patient.

6. The method of claim 1, wherein the information concerning the medication being taken by the patient comprises at least one selected from the group consisting of a name or names of the medication, frequency of administration of the medication, dosage of the medication, a time at which the patient takes the medication, and method of release.

7. The method of claim 1, wherein the information concerning at least one effect of the medication on the patient is a rating of at least one of the patient's energy, interest, focus, concentration, organization, prioritization, productivity, efficiency, effectiveness, quality of work and work product.

8. The method of claim 1, wherein the information concerning characteristics of a current task being performed by the patient, comprises:
an objective description of the task being performed by the patient;
a patient's subjective description of the task being performed by the patient; and
information relating to a setting in which the task is being performed.

9. The method of claim 8, wherein the objective description of the task being performed by the patient is related to at least one of urgency, motor, cognitive, a type of thinking required, learning, memorization, presenting, creativity, complexity, variability and importance.

10. The method of claim 8, wherein the subjective description of the task being performed by the patient is related to at least one of familiarity, whether the task requires new learning, interest, enjoyment, effort, energy and relaxation.

11. The method of claim 8, wherein the setting in which the task is being performed is at least one of home, work, recreation and in transit, degree of support, or degree of distraction.

12. The method of claim 1, wherein receiving information concerning a current baseline of the patient, further comprises:
receiving information concerning how the patient is feeling;
receiving information concerning the patient's inattention;
receiving information concerning the patient's hyperactivity;
receiving information concerning the patient's impulsivity;
receiving information concerning experiences of the patient in the setting; and
receiving information concerning side effects being experienced by the patient.

13. The method of claim 12, wherein the information concerning how the patient is feeling is related to at least one of mood, energy level, alertness, motivation, attention, frustration, anxiety, whether the patient is worried and whether the patient is obsessive.

14. The method of claim 12, wherein the information concerning the patient's inattention is related to at least one of distraction, concentration, attention, carelessness, whether the patient misplaces items, forgetfulness, prioritization, organization, whether the patient avoids complex tasks and whether the patient finishes tasks.

15. The method of claim 12, wherein the information concerning the patient's hyperactivity is related to at least one of activity, whether the patient is fidgety and restlessness.

16. The method of claim 12, wherein the information concerning the patient's impulsivity is related to at least one of whether the patient has difficulty waiting their turn, whether the patient has difficulty relaxing, whether the patient talks too much, whether the patient finishes others sentences and whether the patient interrupts others.

17. The method of claim 12, wherein the information concerning experiences of the patient in the setting is related to at least one of expectations, distractions, interruptions, demands, tension, schedule changes, supportiveness, resources and assistance.

18. The method of claim 12, wherein the side effects being experienced by the patient are related to at least one of appetite, sleep, headache, stomachache, irritability, sedation, fatigue, productivity, motivation and mood.

19. The method of claim 1, wherein the results indicative of the evaluation of effects experienced by the patient relate to at least one of energy, focus, organization, productivity and quality of life.

20. The method of claim 1, wherein the results indicative of side effects experienced by the patient relates to at least one of appetite, insomnia, headache, stomachache, irritability, sedation and fatigue.

21. The method of claim 1, wherein the disorder is selected from the group consisting of an attention-deficit hyperactivity disorder (ADHD), an anxiety disorder, a mood disorder, a thought disorder or substance abuse.

22. The method of claim 1, wherein the disorder is an attention-deficit hyperactivity disorder (ADHD), and the ADHD is selected from the group consisting of ADHD—Predominately Hyperactive-Impulsive Type; ADHD—Predominately Inattentive Type (ADD); and ADHD—Combined Type.

23. A non-transitory computer-readable medium having computer-executable instructions for performing a method comprising:
receiving information concerning a medication being taken by a patient;
receiving information concerning an effect of the medication on the patient;
receiving information concerning characteristics of a current task being performed by the patient;
receiving information concerning a current condition of the patient; and
calculating or providing, by one or more computers, results indicative of an evaluation of effects experienced by the patient upon taking the medication, based on the received information.

24. A method for real-time clinical evaluation of a patient, comprising:
receiving information concerning: (i) a medication being taken by a patient for the treatment of a disorder; (ii) an effect of the medication to the patient; (iii) characteristics of a current task being performed by the patient; and (iv) a current condition of the patient;
calculating or receiving, by one or more computers, results indicative of at least one effect experienced by the patient upon taking the medication. based on the received information; and
analyzing the patient's response to the medication based on the received information and previously obtained patient information.

25. A system for real-time clinical evaluation of a patient, comprising:
(a) a mobile device, configured to:
receive information concerning medication being taken by the patient;
receive information concerning an effect of the medication on the patient;
receive information concerning characteristics of a current task being performed by the patient;
receive information concerning a current condition of the patient;
calculate or provide results indicative of an evaluation of at least one effect experienced by the patient upon taking the medication based on the received information; and
transmit the received information; and
(b) a central computer, comprising a database, configured to:
obtain the received information from the mobile device;
store the received information in the database; and
analyze the patient's response to the medication based on the received information and information previously stored in the database.

26. A method of optimizing the effectiveness of a medication for the treatment of a neurobehavioral or psychiatric disorder in a patient, comprising:
(a) administering a medication at dosage and frequency to a patient for the treatment of a neurobehavioral or psychiatric disorder;
(b) obtaining and storing patient information in a computer database, wherein the patient information relates to at least one selected from the group consisting of the patient's medical history, prior symptoms, developmental history, ability to metabolize the medication (pharmacokinetics), receptor sensitivity to given blood levels of the medication (pharmacodynamics), personality, temperament, current tasks, results from brain imaging, computer Q-EEG information, results of medical, diagnostic or genetic testing, quality of life, use of prescription, over-the-counter or illegal drugs, and hormone levels;
(c) receiving real-time current information sent from a mobile computer device by the patient relating to (i) the medication being taken by the patient; (ii) at least one effect of the medication on the patient; (iii) characteristics of a current task being performed by the patient; and (iv) a current condition of the patient;
(d) evaluating the effectiveness of the medication for the treatment of the neurobehavioral or psychiatric disorder in the patient based on the stored patient information of (b) and the received real-time current information of (c);
(e) modifying, eliminating or maintaining the dosage or frequency of the medication administered to the patient after evaluating the effectiveness of the medication in (d).

27. The method of claim 26, wherein the neurobehavioral or psychiatric disorder is selected from the group consisting of an attention-deficit hyperactivity disorder (ADHD), an anxiety disorder, a mood disorder, a thought disorder or substance abuse.

28. The method of claim 26, wherein the neurobehavioral or psychiatric disorder is an attention-deficit hyperactivity disorder (ADHD), and the ADHD is selected from the group consisting of ADHD—Predominately Hyperactive-Impulsive Type; ADHD—Predominately Inattentive Type (ADD); and ADHD—Combined Type.

29. The method of claim 27, wherein the anxiety disorder is obsessive-compulsive disorder (OCD).

30. The method of claim 27, wherein the mood disorder is selected from the group consisting of depression and bipolar disorder.

31. The method of claim 27, wherein the thought disorder is Aspergers or psychosis and disordered cognitive processing.

32. The method of claim 27, wherein the substance abuse involves previous or current abuse by the patient of a substance selected from the group consisting of alcohol, amphetamines, barbiturates, benzodiazepines, cocaine, synthetic drugs, methaqualone, and opioids.

33. A method for real-time clinical evaluation of the effectiveness of a medication for the treatment of an attention-deficit hyperactivity disorder (ADHD) in a patient, comprising:
(a) treating a patient with a medication for the treatment of an ADHD;
(b) receiving real-time information sent from a computer concerning the medication being taken by the patient;
(c) receiving real-time information sent from a computer concerning at least one effect of the medication on the patient;
(d) receiving real-time information sent from a computer concerning characteristics of a current task being performed by the patient;
(e) receiving real-time information sent from a computer concerning a current condition of the patient; and
(f) determining the effectiveness of medication for the treatment of the ADHD based on the received information of (b)-(e).

34. A method for real-time clinical evaluation of the effectiveness of a medication for the treatment of a neurobehavioral or psychiatric disorder in a patient, comprising:
(a) taking a medication for the treatment of a neurobehavioral or psychiatric disorder;
(b) entering information into a mobile computer device, wherein the information comprises (1) information concerning the medication being taken, and (2) information selected from the group consisting of:
(i) information concerning a benefit or lack of benefit of the medication to the patient;
(ii) information concerning characteristics of a current task being performed by the patient; and
(iii) information concerning a current condition of the patient;
(c) electronically sending the information entered into the mobile computer device to a central computer accessible by a medical practitioner; and
(d) receiving information from the medical practitioner regarding the effectiveness of the medication.

35. The method of claim 34, wherein (b) comprises entering information into a mobile computer device at a regular predetermined time, at least once a week.

36. The method of claim 34, wherein (b) comprises entering information into a mobile computer device at a regular predetermined time, at least daily.

37. The method of claim 34, wherein the neurobehavioral or psychiatric disorder is selected from the group consisting of an attention-deficit hyperactivity disorder (ADHD), an anxiety disorder, a mood disorder, a thought disorder, or substance abuse.

38. The method of claim 37, wherein the neurobehavioral or psychiatric disorder is an ADHD selected from the group consisting of ADHD—Predominately Hyperactive-Impulsive Type; ADHD—Predominately Inattentive Type (ADD); and ADHD—Combined Type.

39. The method of claim 38, wherein the ADHD is adult ADD.

40. The method of claim 38, wherein the ADHD is child ADD or adolescent ADD.

41. The method of claim 38, wherein the medication for the treatment of the ADHD is selected from the group consisting of Vyvanse, Adderall, Concerta, Focalin, Intuniv, or Daytrana.

42. A non-transitory computer-readable medium having computer-executable instructions for performing a method comprising:
(a) receiving information concerning a medication being taken by a patient for the treatment of a neurobehavioral or psychiatric disorder;
(b) receiving information concerning an effect of the medication on the patient;
(c) receiving information concerning characteristics of a current task being performed by the patient;
(d) receiving information concerning a current condition of the patient;
(e) accessing information from a computer database, wherein the accessed information relates to at least one selected from the group consisting of the patient's medical history, prior symptoms, developmental history, ability to metabolize the medication (pharmacokinetics), receptor sensitivity to given blood levels of the medication (pharmacodynamics), personality, temperament, current tasks, results from brain imaging, computer Q-EEG information, results of medical, diagnostic or genetic testing, use of prescription, over-the-counter or illegal drugs, and hormone levels; and
(f) evaluating the effectiveness of medication for the treatment of the neurobehavioral or psychiatric disorder based on the received information of (b)-(d) and the accessed information of (e).

43. A system for real-time clinical evaluation of a patient, comprising:
(a) a mobile device, configured to:
receive information in real-time concerning (i) a medication being taken by a patient for the treatment of a neurobehavioral or psychiatric disorder; (ii) a benefit or lack of benefit of the medication to the patient; (iii) characteristics of a current task being performed by the patient; and (iv) a current condition of the patient; and
transmit the received information; and
(b) a central computer, comprising a database, configured to:
store previously received information in the database relating to at least one selected from the group consisting of the patient's medical history, prior symptoms, developmental history, ability to metabolize the medication (pharmacokinetics), receptor sensitivity to given blood levels of the medication (pharmacodynamics), personality, temperament, current tasks, results from brain imaging, computer Q-EEG information, results of medical, diagnostic or genetic testing, quality of life, and use of prescription, over-the-counter or illegal drugs;
obtain the received real-time information from the mobile device;
store the received real-time information in the database; and
optionally, transmit the previously received information to the mobile device; and
wherein the central computer, and optionally the mobile device, are further configured to:
analyze the effectiveness of the medication for the treatment of the patient of the neurobehavioral or psychiatric disorder based on the received real-time information and the previously received information.

44. A system for real-time clinical evaluation of a patient, comprising a mobile device configured to:
receive information in real-time concerning (i) a medication being taken by a patient for the treatment of a neurobehavioral or psychiatric disorder; (ii) a benefit or lack of benefit of the medication to the patient; (iii) characteristics of a current task being performed by the patient; and (iv) a current condition of the patient; and
transmit the received information;
obtain information from a database of a central computer, wherein the information from the database of the central computer relates to at least one selected from the group consisting of the patient's medical history, prior symptoms, developmental history, ability to metabolize the medication (pharmacokinetics), receptor sensitivity to given blood levels of the medication (pharmacodynamics), personality, temperament, current tasks, results from brain imaging, computer Q-EEG information, results of medical, diagnostic or genetic testing, quality of life, use of prescription, over-the-counter or illegal drugs, and hormone levels; and
analyze the effectiveness of the medication for the treatment of the patient of the neurobehavioral or psychiatric disorder based on the received real-time information and the information obtained from the database of the central computer.

45. A non-transitory computer-readable medium having computer-executable instructions for performing a method of real-time clinical evaluation of a medication for the treatment of a neurobehavioral or psychiatric disorder in a patient, the method comprising:
(A) providing a graphic user interface for accepting user input, wherein the graphic user interface presents the user with options for inputting:
(1) general index information for (i) setting the frequency at which the user will be prompted to provide input, (ii) selecting at least one of a name or type of a medication, a dose, and a time the medication is administered, (iii) selecting at least one of a characteristic of a task, patient interest in the task and patient performance of the task, (iv) selecting at least one of an energy of a patient, mood of a patient and organization ability of a patent, (v) selecting at least one symptom associated with the disorder and (vi) selecting characteristics of the patient environment,
(2) a patient diagnosis or diagnoses specifying at least one of (i) ADD/ADHD—attention disorder, (ii) ODD—oppositional defiant, (iii) mood disorder; (iv) depression, (v) bipolar disorder, (vi) anxiety disorder, (vii) over-reactive emotion, (viii) thought disability, (ix) learning disability and (x) obsessive compulsive disorder,
(3) name or names, or type or types, of at least one current medication of the patient, wherein a dose amount, time administered and whether the medication is a brand name or generic may be specified for each current medication,
(4) survey information comprising at least one of (i) whether the patient is on the same medication as a last rating, (ii) whether the patient takes the same dose of medication as the last rating and (iii) whether the patient takes the medication at the same time as the last rating,
(5) a characterization or rating as to how much the patient's current medication has benefited or improved at least one of the patient's energy, interest, focus, concentration, organization, prioritization, productivity, efficiency and quality of work product,
(6) a characterization or rating as to side effects of the patient's current medication,
(7) a characterization or rating as to how effectively the patient is functioning in at least one of the areas of motivation, productivity, quality of work, mood and relationships,
(8) a characterization or rating as to how much the patient is impaired in at least one of the areas of motivation, productivity, quality of work, mood and relationships,
(9) a characterization or rating as to how much the neurobehavioral or psychiatric disorder interferes with at least one of the patient's motivation, productivity, quality of work, mood and relationships,
(10) a time period during which the patient has difficulty with attention and for each time period, a severity of the patient's inattention and a degree of benefit the patient has obtained from their current medication in dealing with inattention,
(11) a characterization or rating of a task currently being performed by the patient, specifying a degree to which the task is at least one of (i) a motor and cognitive task, (ii) a memorizing and thinking task, (iii) a familiar and new task, (iv) an execution and problem solving task and (v) a planning and presenting task,
(12) a characterization or rating of a task currently being performed by the patient, specifying a degree to which the task is at least one of: (i) urgent, (ii) effort is required, (iii) interesting, (iv) enjoyable, (v) important and (vi) complex,
(13) a characterization or rating of a task currently being performed by the patient, specifying a degree to which the task is at least one of (i) a priority, (ii) requires learning and (iii) is concrete or abstract,
(14) a characterization or rating of the patient, specifying a degree to which the patient is at least one of (i) distracted or attentive, (ii) fatigued or alert, (iii) impulsive or organized, (iv) reckless or deliberate and (v) procrastinating or initiating,
(15) a characterization or rating of the patient, specifying a degree to which the patient is feeling at least one of (i) sad or happy, (ii) anxious or calm, (iii) worried or confident and (iv) passive or motivated,
(16) a severity rating and impact on functioning rating for at least one selected from the group consisting of:
(i) at least one of the following patient diagnoses: (a) ADD/ADHD, (b) depression, (c) bipolar disorder, (d) anxiety, (e) ODD oppositional, (f) over-reactive emotion, (g) learning or reading disability and (h) obsessive compulsive disorder,
(ii) at least one of the following patient symptoms related to depression: (a) low energy, (b) depression or negativity, (c) cannot start and do tasks and (d) self critical or deprecating,
(iii) at least one of the following patient symptoms related to bipolar disorder: (a) variable energy, (b) low energy or staying in bed, (c) high energy or over active and (d) racing thoughts and ideas,
(iv) at least one of the following patient symptoms related to anxiety disorder: (a) anxious, (b) apprehensive or fearful, (c) nervous, (d) expects rejection and (e) avoids social activities,
(v) at least one of the following ADD/ADHD symptoms in the patient: (a) not paying attention, (b) not sustaining attention, (c) not listening, (d) not following-through and (e) not being organized in tasks or activities,
(vi) at least one of the following ADD/ADHD symptoms related to hyperactivity and impulsiveness in the patient: (a) talks excessively, (b) blurts out answers, (c) difficulty waiting turn and (d) interrupts or is intrusive,
(vii) at least one of the following ADD/ADHD symptoms related to the functional impact on the patient: (a) avoids sustained tasks, (b) loses things, (c) is easily distracted and (d) forgets tasks and activities,
(viii) at least one of the following ADD/ADHD symptoms related to the degree of hyperactivity in the patient: (a) fidgets, (b) cannot stay seated, (c) is mentally racing, (d) cannot relax and (e) is feeling driven,
(ix) at least one of the following patient symptoms related to emotional over-reaction: (a) easily upset, frustrated, (b) hard or slow to calm down, (c) often or easily angered, (d) overly self critical and (e) overly critical of others,
(x) at least one of the following patient symptoms related to ODD oppositional, aggressiveness or defiance at home, school or work selected from: (a) failing to do reasonable tasks or assignments, (b) failing to shift from current activity to more important activity when asked, (c) easily angered or loses temper and (d) often stubbornness and resentfulness,
(xi) at least one of the following patient symptoms related to learning difficulty or disability: (a) reading difficulty; (b) difficulty in understanding; (c) math difficulty, (d) being overly self critical and (e) being overly critical of others, and
(xii) at least one of the following patient symptoms related to obsessive compulsive disorder: (a) worried or indecisive, (b) has repetitive thoughts, (c) engages in repetitive actions and (d) engages in rituals or required actions,
(17) a characterization or rating of the patient's environment, specifying at least one of (i) a private office, cubicle, open space or other room at work and (ii) a den or private office, kitchen, living room or other room at home,
(18) a characterization or rating of experiences and expectations in the patient's environment, specifying a degree to which there is at least one of (i) excessive expectations, (ii) a predictable schedule, (iii) distractions, (iv) interruptions and (v) competing demands,
(19) a characterization or rating of morale in the patient's environment, specifying a degree to which the environment is at least one of (i) supportive or loyal, (ii) rewarding, (iii) tense (iv) undermining and (v) other characteristics specified by the patient, and
(20) a characterization or rating of resources in the patient's environment, specifying a degree to which there is at least one of (i) assistance, (ii) equipment and materials, (iii) time for projects, (iv) support for projects and (v) other characteristics specified by the patient;
(B) accepting user input from the user via the graphic user interface; and
(C) either (1) transmitting the accepted user input to one or more computers, or (2) calculating or providing a clinical evaluation and treatment recommendation to the user based on the accepted user input.
